(19) United States
(12) Patent Application Publication
(10) Pub. No.: US 2026/0306710 A1
(43) Pub. Date: Oct. 8, 2026

(54) PHOTOCURABLE HYDROGEL COMPOSITIONS

(71) Applicant: Tympanogen, Inc., Richmond, VA (US)

(72) Inventors: Elaine HORN-RANNEY, Williamsburg, VA (US); Sithara NAIR, Richmond, VA (US); Bethany young, Richmond, VA (US)

(21) Appl. No.: 18/996,805

(22) PCT Filed: Jul. 20, 2023
(86) PCT No.: PCT/US2023/070558

§ 371 Date: Jan. 17, 2025

Related U.S. Application Data
(60) Provisional application No. 63/390,837, filed on Jul. 20, 2022.

(51) Int. Cl. A61L 27/26; A61L 27/52; A61L 27/58

(52) U.S. Cl. CPC A61L 27/26; A61L 27/52; A61L 27/58; A61L 2400/06; A61L 2430/14

(57) ABSTRACT

The present disclosure provides photocurable hydrogel compositions and methods of using in the treatment of tympanic membrane perforations.

### CROSS-REFERENCE TO RELATED APPLICATIONS

The present application claims priority to U.S. Provisional Application No. 63/390,837, filed Jul. 20, 2022, the contents of which are hereby incorporated by reference in their entirety for all purposes.

### GOVERNMENT SUPPORT

This invention was made with government support under Grant No. 1R44DC01855-01 awarded by the National Institute on Deafness and Other Communication Disorders (NIDCD). The government has certain rights in the invention.

### BACKGROUND

Tympanostomy tube (TT) insertion is a very common procedure in children and adults. During TT insertion, a tiny tube is placed into the tympanic membrane (TM) to let fluid leave the middle ear. It also lets air enter the middle ear through the TM. It is most commonly performed due to chronic fluid behind the eardrum to improve hearing and for recurrent middle ear infections to decrease the frequency of infections and direct application of antibiotics topically. In most cases, tympanostomy tubes spontaneously extrude 6 to 18 months after placement with complete repair of the tympanic membrane (TM). However, in about 15% of patients a TM perforation occurs after TT extrusion.

To treat TM perforations, the fibrous tissue around the perforation is abraded to induce an inflammatory response. If the perforation is small such as about 20% or less of the cross-sectional area of the TM, the perforation can be packed with either paper or fat taken from the earlobe. Larger perforations require a more substantial packing material, such as auto grafts of cartilage or fascia, surgical gelatin, or a synthetic polymer patch. All of these therapies require some kind of surgery, and the success rate of the therapy is highly dependent upon the skill of the surgeon.

To date, no photopolymerizable hydrogels have been used as scaffolds for otologic use, in which the semi-gel polymer is applied to the desired site prior to curing. Using a scaffold that is initially a liquid and only forms a scaffold upon exposure to specific conditions is a total departure from current and traditional therapies for repairing TM perforations.

### SUMMARY OF THE DISCLOSURE

The present disclosure provides a pre-lyophilization composition comprising:
(i) a base comprising about 40 wt % to about 80 wt % methacrylated chitosan, about 20 wt % to about 40 wt % hyaluronic acid and about 0.01 wt % to about 0.1 wt % of a photoinitiator; and
(ii) a diluent.

The present disclosure provides a pre-lyophilization composition comprising:
(i) a base comprising about 40 wt % to about 80 wt % methacrylated chitosan, about 20 wt % to about 40 wt % hyaluronic acid and about 0.01 wt % to about 0.5 wt % of a photoinitiator; and
(ii) a diluent.

In embodiments, the photoinitiator is riboflavin.

In embodiments, the composition additionally comprises about 0 wt % to about 12 wt % arginine. In embodiments, the composition additionally comprises about 0 wt % to about 20 wt % arginine.

In embodiments, the methacrylated chitosan is about 30% to about 40% methacrylated. In embodiments, the methacrylated chitosan is about 25% to about 40% methacrylated

The present disclosure provides a lyophilized composition prepared by lyophilization of any of the compositions of the present invention, wherein the lyophilized composition comprises about 0% to about 2% water, and wherein the lyophilized composition can be reconstituted in less than 1 minute.

The present disclosure provides a semi-gel composition prepared by mixing a lyophilized composition of the present disclosure with a diluent comprising water until homogenous; wherein mixing time is less than 60 seconds; wherein the viscosity of the semi-gel composition is about 0.01 kPa*s to about 1.0 kPa*s, as measured by rheometry; wherein the composition comprises about 80 wt % to about 99 wt % water.

The present disclosure provides a cured composition produced by photoirradiation of a semi-gel composition of the present invention, wherein the elastic modulus of the cured composition is about 0.05 kPa to about 20 kPa, as measured by rheometry.

In embodiments, the photoirradiation comprises irradiation of the composition with a wavelength of about 200 nm to about 800 nm, a curing time of about 1 second to about 300 seconds, and a photoirradiation intensity of about 100 mW/cm² to about 1000 mW/cm². In embodiments, the photoirradiation comprises irradiation of the composition with a wavelength of about 200 nm to about 800 nm, a curing time of about 1 second to about 300 seconds, and a photoirradiation intensity of about 100 mW/cm² to about 100000 mW/cm².

The present disclosure provides a kit comprising: a lyophilized composition, a gel dispensing tray, and a light guide.

The present disclosure provides a method of treating a perforation of the tympanic membrane comprising:
(a). applying a semi-gel composition of the present disclosure to the tympanic membrane of a patient in need thereof,
(b). inserting a light guide into the ear canal, and
(c). curing the composition with photoirradiation.

In embodiments, the photoirradiation comprises irradiation of the composition with a wavelength of about 200 nm to about 800 nm, a curing time of about 1 seconds to about 300 seconds and a photoirradiation intensity of about 100 mW/cm² to about 1000 mW/cm². In embodiments, the photoirradiation comprises irradiation of the composition with a wavelength of about 200 nm to about 800 nm, a curing time of about 1 seconds to about 300 seconds and a photoirradiation intensity of about 100 mW/cm² to about 100000 mW/cm².

In embodiments, the present disclosure provides a method of treating a perforation of the tympanic membrane in a patient in need thereof comprising:
(a) mixing a solvent (such as saline) and a lyophilized composition of the present disclosure to provide a semi-gel composition;
(b) administering the semi-gel composition to the perforation site in the tympanic membrane of the patient; and
(c) curing the semi-gel composition.

### BRIEF DESCRIPTION OF THE DRAWINGS

FIGS. 1A-D show the perforation treatment kit components as described in Example 4. Gel pouch with dry reagents (FIG. 1A); gel application kit with dispensing tray (1), light guide (2), and chevron pouch (3) (FIG. 1B); chevron pouch with gel pouch (FIG. 1C); and outer tray with lid, dispensing tray with light guide, gel pouch with lyophilized composition and chevron pouch (FIG. 1D) are shown.

### DETAILED DESCRIPTION OF THE INVENTION

### Definitions

Any term or expression not expressly defined herein shall have its commonly accepted definition understood by those skilled in the art. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation.

The term “semi-gel composition” refers to a composition (comprising at least one polymer) that is capable of being activated to assemble into a more viscous form but that has not yet assembled. In embodiments, the semi-gel composition is sufficiently viscous such that it can be positioned to cover a perforation without falling through to the middle ear cavity. In embodiments, the semi-gel composition may be prepared from a dehydrated composition such as a lyophilized composition.

As used herein, the term “chitosan” means chitosan and its derivatives and analogs (for example methacrylated chitosan). As used herein, the term “hyaluronic acid” means hyaluronic acid, its derivatives and analogs.

The term “treating” with regard to a patient, refers to improving at least one symptom of the patient's disorder. Treating can be improving, or at least partially ameliorating a disorder.

### Compositions

The present disclosure provides semi-gel compositions that, after curing, are useful for the treatment of, among other things, a tympanic membrane rupture in a patient in need thereof, as well as compositions that are useful as intermediates in the manufacture of the semi-gel compositions. Thus, the present disclosure provides pre-lyophilized compositions; lyophilized compositions; semi-gel compositions and cured compositions.

The pre-lyophilized compositions of the present disclosure are flowable mixtures that, upon lyophilization (or a similar technique such as spray-drying) provide shelf-stable lyophilized compositions. The lyophilized compositions of the present disclosure may be reconstituted in a suitable solvent to provide a semi-gel composition that possesses suitable viscosity properties for application to, for example, a rupture in the tympanic membrane of patient in need thereof. After application to a patient in need thereof, the semi-gel composition may be cured using methods that are disclosed herein to provide a cured composition (or scaffold).

In embodiments, compositions of the present disclosure comprise ingredients that are biocompatible and suitable for use in the ear, e.g., the external auditory canal, middle ear, or both. In embodiments, the ingredients are chosen such that the resultant polymer matrix is biodegradable and may be processed and cleared by the body, e.g., by the kidneys.

The compositions according to this disclosure are initially a semi-gel composition for administration to a desired site in a patient's ear and are configured to cure into a polymer matrix in the ear of a patient upon activation. Accordingly, the semi-gel composition includes one or more polymers capable of selectively assembling into a polymer matrix at a desired time under specific conditions (for example conditions found in the ear, such as the middle ear), and a diluent. In embodiments, the semi-gel composition includes an initiator (e.g., a photoinitiator), and the semi-gel composition is cured by exposing the composition to a condition (e.g., light) thereby activating the initiator and resulting in a polymer matrix. In embodiments, changes in, light density, pH, temperature, or ion concentration initiate spontaneous assembly of the polymer matrix. In embodiments, the polymers are functionalized to give them crosslink ability (e.g., methacrylated) upon activation of the initiator (e.g., riboflavin).

In embodiments, the semi-gel composition is designed to cure into a polymer matrix or scaffold having a porosity and average pore size compatible with loading the resultant polymer matrix or scaffold with a therapeutic agent. In embodiments, the porosity and average pore size are chosen to provide a desired time-dependent release profile for the loaded therapeutic agent.

In embodiments, the polymer matrix is a scaffold having sufficient stiffness and/or tackiness (for example as a result of functional groups providing cell adhesion domains) such that epithelial cells in the TM can migrate into and/or onto the scaffold and adhere and/or grow.

In embodiments, the polymer matrix or scaffold eventually degrades in situ, i.e., is biodegradable, for example after the epithelial cells have grown sufficiently to repair the perforation.

### Pre-Lyophilized Compositions

The present disclosure provides a pre-lyophilized composition comprising: (i) a base comprising methacrylated chitosan, hyaluronic acid and a photoinitiator; and (ii) a diluent. In embodiments, the diluent comprises water.

In embodiments, the pre-lyophilized compositions provide, upon lyophilization (or a similar technique such as spray-drying), a shelf-stable lyophilized composition.

In embodiments, the present disclosure provides a pre-lyophilized composition comprising:
(i) a base comprising about 40 wt % to about 80 wt % methacrylated, about 20 wt % to about 40 wt % hyaluronic acid, about 0.01 wt % to about 0.1 wt % of a photoinitiator and 0 wt % to about 20 wt % arginine; and
(ii) a diluent.

In embodiments, the present disclosure provides a pre-lyophilized composition comprising:
(i) a base comprising about 40 wt % to about 80 wt % methacrylated chitosan, about 20 wt % to about 40 wt % hyaluronic acid, about 0.01 wt % to about 0.5 wt % of a photoinitiator and 0 wt % to about 20 wt % arginine; and
(ii) a diluent.

In embodiments, the base comprises about 30 wt % to about 90 wt % methacrylated chitosan, e.g. about 30 wt %, about 32 wt %, about 34 wt %, about 36 wt %, about 38 wt %, about 40 wt %, about 42 wt %, about 44 wt %, about 46 wt %, about 48 wt %, about 50 wt %, about 52 wt %, about 54 wt %, about 56 wt %, about 58 wt %, about 60 wt %, about 62 wt %, about 64 wt %, about 66 wt %, about 68 wt %, about 70 wt %, about 72 wt %, about 74 wt %, about 76 wt %, about 78 wt %, about 80 wt %, about 82 wt %, about 84 wt %, about 86 wt %, about 88 wt %, or about 90 wt %, including all ranges and values in between. In embodiments, the composition comprises about 40 wt % to about 80 wt % methacrylated chitosan. In embodiments, the composition comprises about 60 wt % methacrylated chitosan.

In embodiments, the methacrylated chitosan is about 10% to about 60% methacrylated, e.g. about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 28%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%, including all ranges and values in between. In embodiments, the methacrylated chitosan is about 21% to about 32% methacrylated. In embodiments, the methacrylated chitosan is about 21% to about 34% methacrylated. In embodiments, the methacrylated chitosan is about 21% to about 36% methacrylated. In embodiments, the methacrylated chitosan is about 25% to about 40% methacrylated. In embodiments, the methacrylated chitosan is about 30% methacrylated.

In embodiments, the molecular weight of the chitosan may range from about 100 to about 1,000,000 g/mol. In embodiments, deacetylation of the chitosan ranges from about 0 to about 100%.

In embodiments, the base comprises about 10 wt % to about 50 wt % hyaluronic acid, e.g., about 10 wt %, about 12 wt %, about 14 wt %, about 16 wt %, about 18 wt %, about 20 wt %, about 22 wt %, about 24 wt %, about 26 wt %, about 28 wt %, about 30 wt %, about 32 wt %, about 34 wt %, about 36 wt %, about 38 wt %, about 40 wt %, about 42 wt %, about 44 wt %, about 46 wt %, about 48 wt %, or about 50 wt %, including all ranges and values in between. In embodiments, the composition comprises about 20 wt % to about 40 wt % hyaluronic acid. In embodiments, the composition comprises about 30 wt % hyaluronic acid.

In embodiments, the molecular weight of the hyaluronic acid may range from about 100 g/mol to about 1,000,000 g/mol.

A “photoinitiator” typically includes an agent that forms free radicals when illuminated by light of appropriate wavelengths. In embodiments, the photoinitiator is an aromatic carbonyl compound (e.g., benzoin derivatives, benziketals, acetophenone derivatives, hydroxyalkylphenones) or an aromatic ketone (e.g., benzophenone and thioxanthone). In embodiments, the photoinitiator is benzophenone, dimethoxyphenyl acetophenone, 2,2-dimethoxy-2-phenylacetophenone and 2,2-diethoxyacetophenone, 1-[4-(2-hydroxyethoxy)-phenyl]-2-hydroxy-2-methyl-1-propane-1-one, ethyl eosin, eosin Y, fluorescein, 2,2-dimethoxy-2-phenylacetophenone, 2-methyl-2-phenylacetonphenone, 12959, camphorquinone, rose bengal, methylene blue, erythosin, phloxime, thionine, riboflavin, and methyl green. Still other photoinitiators comprise 1-(4-Fluorphenyl)-2-methyl-2-morpholino-1-propanone, 1,7-bis(9-acridinyl)heptane, 1-Chloro-4-propoxythioxanthone, 1-Hydroxy cyclohexyl phenyl ketone, 2,2-Di ethoxy acetophenone, 2,3,4,4′-Tetrahydroxy Benzophenone, 2,3,4-Trihydroxybenzophenone, 2,4,6-Trimethyl benzoyl diphenyl phosphine oxide, 2,4,6-Trimethylbenzophenone, 2/4-Diethylthioxanthone, 2/4-Isopropylthioxanthone, 2-Benzyl-2-(dimethylamino)-1-[4-(4-morpholinyl)phenyl]-1-butanone, 2-Chlorothioxanthone, 2-Dimethyl-aminoethylbenzoate, 2-Ethylhexyl-4-dimethylaminobenzoate, 2-Hydroxy-2-methyl-phenyl-propan-1-one, 2-Hydroxy-4′-hydroxyethoxy-2-methylpropiophenone, 2-Isopropylthioxanthone, 2-Methyl benzophenone, 2-Methyl-1-[4-(methylthio)phenyl]-2-morpholinopropanone-1,4-(4-Methylphen-ylthiophenyl)-phenylmethanone, 4,4′-Difluoro benzophenone, 4,4′-Dimethoxy benzophenone, 4-Chloro benzophenone, 4-Methyl acetophenone, 4-Methyl benzophenone, 4-Phenylbenzophenone, Benzil dimethyl ketal, Benzophenone, Benzophenone hydrazone, Bis(p-tolyl) iodonium hexafluorophosphate, Dimethyl Sebacate, Diphenyl lodonium Hexafluorophosphate, Ethyl (2,4,6-trimethylbenzoyl)phenylphosphinate, Ethyl-4-(dimethylamino)benzoate, Methyl o-benzoyl benzoate, Methyl phenyl glyoxylate, N,N,N′,N′-Tetraethyl-4,4-diaminobenzophenone, Phenyltribromomethylsulphone, acylphosphine oxide (APO) and bisacylphosphine oxide (BAPO), 1-[4-(2-Hydroxyethoxy)-phenyl]-2-hydroxy-2-methyl-1-propane-1-one, 2,2-Dimethoxy-1,2-diphenylethan-1-one, hydroxy-cyclohexyl-phenyl-ketone, methylbenzoylformate, oxy-phenyl-acetic acid 2-[2 oxo-2 phenyl-acetoxy-ethoxy]ethyl ester, oxy-phenyl-acetic2-[2-hydroxy-ethoxy]-ethyl ester, alpha-dimethoxy-alpha-phenylacetophenone, 2-Benzyl-2-(dimethylamino)-1-[4-(4-morpholinyl)phenyl]-1-butanone, diphenyl (2,4,6-trimethylbenzoyl)-phosphine oxide, phosphine oxide, bis(eta 5-2,4-cyclopentadien-1-yl), bis[2,6-difluoro-3-(1H-pyrrol-1-yl)phenyl]titanium, Iodonium, (4-methylphenyl)[4-(2-methylpropyl)phenyl]-hexafluorophosphate(1-), bis(2,6-dimethoxybenz-oyl)-2,4,4-trimethyl pentylphosphineoxide, or derivatives thereof.

In embodiments, the photoinitiator is riboflavin. In embodiments, the riboflavin is riboflavin 5′ monophosphate sodium salt.

In embodiments, the base comprises about 0.01 wt % to about 0.10 wt % of a photoinitiator. In embodiments, the base comprises about 0.01 wt % to about 0.5 wt % of a photoinitiator, e.g. about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.10 wt %, about 0.11 wt %, 0.12 wt %, about 0.13 wt %, about 0.14 wt %, about 0.15 wt %, about 0.16 wt %, about 0.17 wt %, about 0.18 wt %, about 0.19 wt %, about 0.20 wt %, about 0.21 wt %, 0.22 wt %, about 0.23 wt %, about 0.24 wt %, about 0.25 wt %, about 0.26 wt %, about 0.27 wt %, about 0.28 wt %, about 0.29 wt %, about 0.30 wt %, about 0.31 wt %, about 0.32 wt %, about 0.33 wt %, about 0.34 wt %, about 0.35 wt %, about 0.36 wt %, about 0.37 wt %, about 0.38 wt %, about 0.39 wt %, about 0.40 wt %, about 0.41 wt %, 0.42 wt %, about 0.43 wt %, about 0.44 wt %, about 0.45 wt %, about 0.46 wt %, about 0.47 wt %, about 0.48 wt %, about 0.49 wt % or about 0.5 wt %, including all ranges and values in between. In embodiments, the composition comprises about 0.08 wt % to about 0.016 wt % of a photoinitiator. In embodiments, the composition comprises about 0.08 wt % to about 0.40 wt % of a photoinitiator. In embodiments, the composition comprises about 0.08 wt % of a photoinitiator. In embodiments, the composition comprises about 0.50 wt % of a photoinitiator. In embodiments, the composition comprises about 0.12 wt % of a photoinitiator. In embodiments, the composition comprises about 0.40 wt % of a photoinitiator.

Applicant found that adding arginine to a composition of the present disclosure improves gel formation and the distribution of other reagents within gel. In embodiments, arginine acts as co-initiator for crosslinking.

In embodiments, the base comprises arginine. In embodiments, the arginine is L-arginine. In embodiments, the base comprises about 0 wt % to about 20 wt % arginine, e.g. about 0 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt %, including all ranges and values in between. In embodiments, the base comprises about 6 wt % to about 12 wt % arginine. In embodiments, the base comprises about 6 wt % arginine. In embodiments, the base comprises about 9 wt % arginine.

In embodiment, the base comprises an amine catalyst, e.g., Ethyl 4-dimethylaminobenzoate (EDMAB).

In embodiment, the base comprises a free-radical inhibitor, e.g., 2,6-di-tert butyl-4-methyl phenol (BHT).

In embodiments, the diluent is about 0 wt % to about 99 wt % of the composition. In embodiments, the diluent is about 1 wt % to about 99 wt % of the composition, e.g. about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, about 31 wt %, about 32 wt %, about 33 wt %, about 34 wt %, about 35 wt %, about 36 wt %, about 37 wt %, about 38 wt %, about 39 wt %, about 40 wt %, about 41 wt %, about 42 wt %, about 43 wt %, about 44 wt %, about 45 wt %, about 46 wt %, about 47 wt %, about 48 wt %, about 49 wt %, about 50 wt %, about 51 wt %, about 52 wt %, about 53 wt %, about 54 wt %, about 55 wt %, about 56 wt %, about 57 wt %, about 58 wt %, about 59 wt %, about 60 wt %, about 61 wt %, about 62 wt %, about 63 wt %, about 64 wt %, about 65 wt %, about 66 wt %, about 67 wt %, about 68 wt %, about 69 wt %, about 70 wt %, about 71 wt %, about 72 wt %, about 73 wt %, about 74 wt %, about 75 wt %, about 76 wt %, about 77 wt %, about 78 wt %, about 79 wt %, about 80 wt %, about 81 wt %, about 82 wt %, about 83 wt %, about 84 wt %, about 85 wt %, about 86 wt %, about 87 wt %, about 88 wt %, about 89 wt %, about 90 wt %, about 91 wt %, about 92 wt %, about 93 wt %, about 94 wt %, about 95 wt %, about 96 wt %, about 97 wt %, about 98 wt %, or about 99 wt %, including all ranges and values in between. In embodiments, the diluent is about 0 wt % to about 95 wt % of the composition. In embodiments, the diluent is about 1 wt % to about 95 wt % of the composition. In embodiments, the diluent is about 97 wt % of the composition. In embodiments, the diluent is about 1 wt % of the composition. In embodiments, the diluent is about 2 wt % of the composition. In embodiments, the diluent comprises water. In embodiments, the diluent comprises water and sodium chloride. In embodiments, the diluent comprises water and phosphate buffer. In embodiments, the diluent comprises water, sodium chloride and phosphate buffer. In embodiments the diluent is 0.9% sodium chloride in water (i.e., saline).

### Lyophilized Composition

The pre-lyophilized compositions of the present disclosure may be lyophilized to produce a lyophilized composition. Without being limited by theory, lyophilization increases the shelf-life of compositions of the present disclosure. In embodiments, lyophilization of the composition provides a shelf life of greater than 1 year. In embodiments, lyophilization of the composition provides a shelf life of about 1 year.

In embodiments, the lyophilized composition is porous to allow a faster reconstitution time. In embodiments, the porosity is introduced by lyophilization, gassing or introduction of ice particles while freezing. In embodiments, the lyophilization parameters are critical to prevent the collapse of the material during the freeze drying process. Insufficient drying results in collapse of the gel creating dense, hard material, which becomes very difficult to reconstitute and has very poor gel consistency.

In embodiments, the lyophilized composition comprises water, methacrylated chitosan, hyaluronic acid, and a photoinitiator. In embodiments, the lyophilized composition further comprises arginine. In embodiments, the arginine is L-arginine. In embodiments, the photoinitiator is riboflavin. In embodiments, the riboflavin is riboflavin 5′ monophosphate sodium salt. In embodiments, the present disclosure provides a lyophilized composition comprising about 0 wt % to about 10 wt % water, 50 wt % to about 65 wt % methacrylated chitosan, 25 wt % to about 35 wt % hyaluronic acid, 0.10 wt % to about 0.15 wt % riboflavin, and 0 wt % to about 10 wt % arginine. In embodiments, the present disclosure provides a lyophilized composition comprising about 0 wt % to about 10 wt % water, 40 wt % to about 80 wt % methacrylated chitosan, 20 wt % to about 40 wt % hyaluronic acid, 0.01 wt % to about 0.5 wt % riboflavin, and 0 wt % to about 20 wt % arginine.

In embodiments, the lyophilized composition comprises about 0 wt % to about 10 wt % water, e.g. about 0 wt %, about 0.5 wt %, about 1.0 wt %, about 1.5 wt %, about 2.0 wt %, about 2.5 wt %, about 3.0 wt %, about 3.5 wt %, about 4.0 wt %, about 4.5 wt %, about 5.0 wt %, about 5.5 wt %, about 6.0 wt %, about 6.5 wt %, about 7.0 wt %, about 7.5 wt %, about 8.0 wt %, about 8.5 wt %, about 9.0 wt %, about 9.5 wt %, or about 10.0 wt %, including all ranges and values in between. In embodiments, the lyophilized composition comprises about 0 wt % to about 2 wt % water. In embodiments, the lyophilized composition comprises about 0 wt % water. In embodiments, the lyophilized composition comprises about 1 wt % water. In embodiments, the lyophilized composition comprises about 2 wt % water.

In embodiments, the lyophilized composition comprises about 50 wt % to about 65 wt % methacrylated chitosan. In embodiments, the lyophilized composition comprises about 30 wt % to about 90 wt % methacrylated chitosan, e.g., about 30 wt %, about 31 wt %, about 32 wt %, about 33 wt %, about 34 wt %, about 35 wt %, about 36 wt %, about 37 wt %, about 38 wt %, about 39 wt %, about 40 wt %, about 41 wt %, about 42 wt %, about 43 wt %, about 44 wt %, about 45 wt %, about 46 wt %, about 47 wt %, about 48 wt %, about 49 wt %, about 50 wt %, about 51 wt %, about 52 wt %, about 53 wt %, about 54 wt %, about 55 wt %, about 56 wt %, about 57 wt %, about 58 wt %, about 59 wt %, about 60 wt %, about 61 wt %, about 62 wt %, about 63 wt %, about 64 wt %, about 65 wt %, about 66 wt %, about 67 wt %, about 68 wt %, about 69 wt %, about 70 wt %, about 71 wt %, about 72 wt %, about 73 wt %, about 74 wt %, about 75 wt %, about 76 wt %, about 77 wt %, about 78 wt %, about 79 wt %, about 80 wt %, about 81 wt %, about 82 wt %, about 83 wt %, about 84 wt %, about 85 wt %, about 86 wt %, about 87 wt %, about 88 wt %, about 89 wt %, or about 90 wt %, including all ranges and values in between. In embodiments, the lyophilized composition comprises about 59 wt % methacrylated chitosan. In embodiments, the lyophilized composition comprises about 60 wt % methacrylated chitosan. In embodiments, the lyophilized composition comprises about 61 wt % methacrylated chitosan. In embodiments, the lyophilized composition comprises about 40 wt % to about 80 wt % methacrylated chitosan. In embodiments, the lyophilized composition comprises about 50 wt % to about 65 wt % methacrylated chitosan.

In embodiments, the lyophilized composition comprises about 25 wt % to about 35 wt % hyaluronic acid. In embodiments, the lyophilized composition comprises about 10 wt % to about 50 wt % hyaluronic acid e.g. about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, about 31 wt %, about 32 wt %, about 33 wt %, about 34 wt %, about 35 wt %, about 36 wt %, about 37 wt %, about 38 wt %, about 39 wt %, about 40 wt %, about 41 wt %, about 42 wt %, about 43 wt %, about 44 wt %, about 45 wt %, about 46 wt %, about 47 wt %, about 48 wt %, about 49 wt %, or about 50 wt %, including all ranges and values in between. In embodiments, the lyophilized composition comprises about 29 wt % hyaluronic acid. In embodiments, the lyophilized composition comprises about 30 wt % hyaluronic acid. In embodiments, the lyophilized composition comprises about 31 wt % hyaluronic acid. In embodiments, the lyophilized composition comprises about 20 wt % to about 40 wt % hyaluronic acid. In embodiments, the lyophilized composition comprises about 25 wt % to about 35 wt % hyaluronic acid.

In embodiments, the photoinitiator is an aromatic carbonyl compound (e.g., benzoin derivatives, benziketals, acetophenone derivatives, hydroxyalkylphenones) or an aromatic ketone (e.g., benzophenone and thioxanthone). In embodiments, the photoinitiator is benzophenone, dimethoxyphenyl acetophenone, 2,2-dimethoxy-2-phenylacetophenone and 2,2-diethoxyacetophenone, 1-[4-(2-hydroxyethoxy)-phenyl]-2-hydroxy-2-methyl-1-propane-1-one, ethyl eosin, eosin Y, fluorescein, 2,2-dimethoxy-2-phenylacetophenone, 2-methyl-2-phenylacetonphenone, 12959, camphorquinone, rose bengal, methylene blue, erythosin, phloxime, thionine, riboflavin, and methyl green. Still other photoinitiators comprise 1-(4-Fluorphenyl)-2-methyl-2-morpholino-1-propanone, 1,7-bis(9-acridinyl)heptane, 1-Chloro-4-propoxythioxanthone, 1-Hydroxy cyclohexyl phenyl ketone, 2,2-Di ethoxy acetophenone, 2,3,4,4′-Tetrahydroxy Benzophenone, 2,3,4-Trihydroxybenzophenone, 2,4,6-Trimethyl benzoyl diphenyl phosphine oxide, 2,4,6-Trimethylbenzophenone, 2/4-Diethylthioxanthone, 2/4-Isopropylthioxanthone, 2-Benzyl-2-(dimethylamino)-1-[4-(4-morpholinyl)phenyl]-1-butanone, 2-Chlorothioxanthone, 2-Dimethyl-aminoethylbenzoate, 2-Ethylhexyl-4-dimethylaminobenzoate, 2-Hydroxy-2-methyl-phenyl-propan-1-one, 2-Hydroxy-4′-hydroxyethoxy-2-methylpropiophenone, 2-Isopropylthioxanthone, 2-Methyl benzophenone, 2-Methyl-1-[4-(methylthio)phenyl]-2-morpholinopropanone-1,4-(4-Methylphen-ylthiophenyl)-phenylmethanone, 4,4′-Difluoro benzophenone, 4,4′-Dimethoxy benzophenone, 4-Chloro benzophenone, 4-Methyl acetophenone, 4-Methyl benzophenone, 4-Phenylbenzophenone, Benzil dimethyl ketal, Benzophenone, Benzophenone hydrazone, Bis(p-tolyl) iodonium hexafluorophosphate, Dimethyl Sebacate, Diphenyl lodonium Hexafluorophosphate, Ethyl (2,4,6-trimethylbenzoyl)phenylphosphinate, Ethyl-4-(dimethylamino)benzoate, Methyl o-benzoyl benzoate, Methyl phenyl glyoxylate, N,N,N′,N′-Tetraethyl-4,4-diaminobenzophenone, Phenyltribromomethylsulphone, acylphosphine oxide (APO) and bisacylphosphine oxide (BAPO), 1-[4-(2-Hydroxyethoxy)-phenyl]-2-hydroxy-2-methyl-1-propane-1-one, 2,2-Dimethoxy-1,2-diphenylethan-1-one, hydroxy-cyclohexyl-phenyl-ketone, methylbenzoylformate, oxy-phenyl-acetic acid 2-[2 oxo-2 phenyl-acetoxy-ethoxy]ethyl ester, oxy-phenyl-acetic2-[2-hydroxy-ethoxy]-ethyl ester, alpha-dimethoxy-alpha-phenylacetophenone, 2-Benzyl-2-(dimethylamino)-1-[4-(4-morpholinyl)phenyl]-1-butanone, diphenyl (2,4,6-trimethylbenzoyl)-phosphine oxide, phosphine oxide, bis(eta 5-2,4-cyclopentadien-1-yl), bis[2,6-difluoro-3-(1H-pyrrol-1-yl)phenyl]titanium, Iodonium, (4-methylphenyl)[4-(2-methylpropyl)phenyl]-hexafluorophosphate(1-), bis(2,6-dimethoxybenz-oyl)-2,4,4-trimethyl pentylphosphineoxide, or derivatives thereof.

In embodiments, the photoinitiator is riboflavin. In embodiments, the riboflavin is riboflavin 5′ monophosphate sodium salt. In embodiments, the lyophilized composition comprises about 0.10 wt % to about 0.15 wt % riboflavin. In embodiments, the lyophilized composition comprises about 0.10 wt % to about 0.7 wt % riboflavin, e.g., about 0.10 wt %, about 0.11 wt %, about 0.12 wt %, about 0.13 wt %, about 0.14 wt %, about 0.15 wt %, about 0.16 wt %, about 0.17 wt %, about 0.18 wt %, about 0.19 wt %, about 0.20 wt %, about 0.21 wt %, 0.22 wt %, about 0.23 wt %, about 0.24 wt %, about 0.25 wt %, about 0.26 wt %, about 0.27 wt %, about 0.28 wt %, about 0.29 wt %, about 0.30 wt %, about 0.31 wt %, 0.32 wt %, about 0.33 wt %, about 0.34 wt %, about 0.35 wt %, about 0.36 wt %, about 0.37 wt %, about 0.38 wt %, about 0.39 wt %, about 0.40 wt %, about 0.41 wt %, 0.42 wt %, about 0.43 wt %, about 0.44 wt %, about 0.45 wt %, about 0.46 wt %, about 0.47 wt %, about 0.48 wt %, about 0.49 wt %, about 0.50 wt %, about 0.51 wt %, 0.52 wt %, about 0.53 wt %, about 0.54 wt %, about 0.55 wt %, about 0.56 wt %, about 0.57 wt %, about 0.58 wt %, about 0.59 wt %, about 0.60 wt %, about 0.61 wt %, 0.62 wt %, about 0.63 wt %, about 0.64 wt %, about 0.65 wt %, about 0.66 wt %, about 0.67 wt %, about 0.68 wt %, about 0.69 wt %, or about 0.7 wt %, including all ranges and values in between. In embodiments, the riboflavin is riboflavin 5′ monophosphate sodium salt. In embodiments, the lyophilized comprises about 0.11 wt % riboflavin. In embodiments, the lyophilized composition comprises about 0.12 wt % riboflavin. In embodiments, the lyophilized composition comprises about 0.13 wt % riboflavin. In embodiments, the lyophilized composition comprises about 0.01 wt % to about 0.5 wt % riboflavin. In embodiments, the lyophilized composition comprises about 0.01 wt % to about 0.15 wt % riboflavin.

In embodiments, the lyophilized composition comprises about 0 wt % to about 10 wt % arginine. In embodiments, the lyophilized composition comprises about 0 wt % to about 30 wt % arginine, e.g. about 0 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, or about 30 wt %, including all ranges and values in between. In embodiments, the lyophilized composition comprises about 8 wt % arginine. In embodiments, the arginine is L-arginine. In embodiments, the lyophilized composition comprises about 9 wt % arginine. In embodiments, the lyophilized composition comprises about 10 wt % arginine. In embodiments, the lyophilized composition comprises about 0 wt % to about 10 wt % arginine. In embodiments, the lyophilized composition comprises about 0 wt % to about 20 wt % arginine. In embodiments, the arginine is L-arginine.

In embodiments, the composition is a lyophilized composition prepared by lyophilization of any one of the pre-lyophilization compositions of the present disclosure, comprising about 0 wt % to about 2 wt % water, wherein the lyophilized composition can be reconstituted in less than 60 seconds to provide a semi-gel composition of the present disclosure.

In embodiments, the lyophilized composition comprises about 1 wt % water, about 60 wt % methacrylated chitosan, about 30 wt % hyaluronic acid, and about 9 wt % L-arginine, and 0.12 wt % riboflavin 5′ monophosphate sodium salt.

In embodiments, the lyophilized composition is sterilized, wherein the sterilization is achieved with ionizing radiation. In embodiments, the ionizing radiation is electron beam radiation. In embodiments, the ionizing radiation is x-ray radiation. In embodiments, the ionizing radiation is gamma radiation.

In embodiments, sterilization partially cures the composition. In embodiments, sterilization is required to fully cure the composition. In embodiments, e-beam radiation partially cures the gel. In embodiments, x-ray radiation partially cures the gel. In embodiments, the lyophilized composition is sterilized, wherein the sterilization is achieved with ionizing radiation. In embodiments, the ionizing radiation is gamma radiation. In embodiments, gamma irradiation partially cures the composition.

In embodiments, the dose of ionizing radiation is about 10 kGy to about 70 kGy, about 10 kGy, about 12 kGy, about 14 kGy, about 16 kGy, about 18 kGy, about 20 kGy, about 22 kGy, about 24 kGy, about 25 kGy, about 26 kGy, about 28 kGy, about 30 kGy, about 32 kGy, about 34 kGy, about 36 kGy, about 38 kGy, about 40 kGy, about 42 kGy, about 44 kGy, about 46 kGy, about 48 kGy, about 50 kGy, about 52 kGy, about 54 kGy, about 56 kGy, about 58 kGy, about 60 kGy, about 62 kGy, about 64 kGy, about 66 kGy, about 68 kGy, or about 70 kGy, including all ranges and values in between. In embodiments, the dose of ionizing radiation is about 25 kGy.

In embodiments, after sterilization, there is a log reduction of between about 2 and about 4 in colony forming units, e.g. about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, or about 5.0, including all ranges and values in between. In embodiments, after sterilization, there is a log reduction of between about 3.4 and about 3.6 in colony forming units. In embodiments, after sterilization, there is a log reduction of about 3.4 in colony forming units.

### Semi-Gel Composition

In embodiments, a lyophilized composition of the present disclosure is reconstituted to provide a semi-gel composition. In embodiments, the semi-gel composition is prepared by mixing a lyophilized composition of the present disclosure with a diluent until homogenous.

As used herein, the term “reconstitution time” means the mixing time required during reconstitution to form a homogenous mixture. In embodiments, the mixing time is about 5 seconds to about 120 seconds, e.g., about 1 sec, about 2 sec, about 3 sec, about 4 sec, about 5 sec, about 6 sec, about 7 sec, about 8 sec, about 9 sec, about 10 sec, about 12 sec, about 14 sec, about 16 sec, about 18 sec, about 20 sec, about 22 sec, about 24 sec, about 26 sec, about 28 sec, about 30 sec, about 32 sec, about 34 sec, about 36 sec, about 38 sec, about 40 sec, about 42 sec, about 44 sec, about 46 sec, about 48 sec, about 50 sec, about 52 sec, about 54 sec, about 56 sec, about 58 sec, about 60 sec, about 65 sec, about 70 sec, about 75 sec, about 80 sec, about 85 sec, about 90 sec, about 95 sec, about 100 sec, about 110 sec, or about 120 sec, including all values or ranges therebetween. In embodiments, reconstitution takes about 30 seconds. In embodiments, reconstitution takes less than about 60 seconds. In embodiments, reconstitution takes less than about 90 seconds.

In embodiments, the semi-gel composition comprises a diluent, methacrylated chitosan, hyaluronic acid, and a photoinitiator. In embodiments, the semi-gel composition further comprises arginine. In embodiments, the arginine is L-arginine. In embodiments, the photoinitiator is riboflavin. In embodiments, the riboflavin is riboflavin 5′ monophosphate sodium salt. In embodiments, the present disclosure provides a semi-gel composition comprising about 80 wt % to about 99 wt % of a diluent, about 1 wt % to about 3 wt % methacrylated chitosan, about 0.5 wt % to about 2 wt % hyaluronic acid, about 0.0010 wt % to about 0.01 wt % of a photoinitiator, and about 0 wt % to about 0.5 wt % arginine.

In embodiments, the final concentration of diluent in the semi-gel composition is about 80 wt % to about 99 wt %, e.g., about 80 wt %, about 81 wt %, about 82 wt %, about 83 wt %, about 84 wt %, about 85 wt %, about 86 wt %, about 87 wt %, about 88 wt %, about 89 wt %, about 90 wt %, about 91 wt %, about 92 wt %, about 93 wt %, about 94 wt %, about 95 wt %, about 96 wt %, about 97 wt %, about 98 wt %, or about 99 wt %, including all ranges and values in between. In embodiments, the final concentration of diluent in the semi-gel composition is about 96 wt %. In embodiments, the final concentration of diluent in the semi-gel composition is about 97 wt %. In embodiments, the final concentration of diluent in the semi-gel composition is about 98 wt %. In embodiments, the diluent comprises water. In embodiments, the diluent comprises water and sodium chloride. In embodiments, the diluent comprises water and phosphate buffer. In embodiments, the diluent comprises water, sodium chloride and phosphate buffer. In embodiments the diluent is 0.9% sodium chloride in water (i.e., saline).

In embodiments, the semi-gel composition is prepared by mixing any one of the compositions of the disclosure with a diluent until homogenous; wherein the final concentration of diluent is about 97 wt %; wherein mixing time is less than about 60 seconds; wherein the viscosity of the semi-gel composition is about 0.01 kPa*s to about 1.0 kPa*s, as measured by rheometry.

In embodiments, the semi-gel composition comprises about 80 wt % to about 99 wt % water, e.g about 80 wt %, about 81 wt %, about 82 wt %, about 83 wt %, about 84 wt %, about 85 wt %, about 86 wt %, about 87 wt %, about 88 wt %, about 89 wt %, about 90 wt %, about 91 wt %, about 92 wt %, about 93 wt %, about 94 wt %, about 95 wt %, about 96 wt %, about 97 wt %, about 98 wt %, or about 99 wt %, including all ranges and values in between. In embodiments, the semi-gel composition comprises about 90 wt % to about 97 wt % water. In embodiments, the semi-gel composition comprises about 97 wt % water.

In embodiments, the semi-gel composition comprises about 1 wt % to about 3 wt % methacrylated chitosan, e.g. about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2 wt %, about 2.1 wt %, about 2.2 wt %, about 2.3 wt %, about 2.4 wt %, about 2.5 wt %, about 2.6 wt %, about 2.7 wt %, about 2.8 wt %, about 2.9 wt %, or about 3 wt %, including all ranges and values in between. In embodiments, the semi-gel composition comprises about 2 wt % methacrylated chitosan. In embodiments, the methacrylated chitosan is about 25% to about 40% methacrylated.

In embodiments, the semi-gel composition comprises about 0.5 wt % to about 2 wt % hyaluronic acid, e.g. about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, or about 2 wt %, including all ranges and values in between. In embodiments, the semi-gel composition comprises about 1 wt % hyaluronic acid.

In embodiments, the photoinitiator is an aromatic carbonyl compound (e.g., benzoin derivatives, benziketals, acetophenone derivatives, hydroxyalkylphenones) or an aromatic ketone (e.g., benzophenone and thioxanthone). In embodiments, the photoinitiator is benzophenone, dimethoxyphenyl acetophenone, 2,2-dimethoxy-2-phenylacetophenone and 2,2-diethoxyacetophenone, 1-[4-(2-hydroxyethoxy)-phenyl]-2-hydroxy-2-methyl-1-propane-1-one, ethyl eosin, eosin Y, fluorescein, 2,2-dimethoxy-2-phenylacetophenone, 2-methyl-2-phenylacetonphenone, 12959, camphorquinone, rose bengal, methylene blue, erythosin, phloxime, thionine, riboflavin, and methyl green. Still other photoinitiators comprise 1-(4-Fluorphenyl)-2-methyl-2-morpholino-1-propanone, 1,7-bis(9-acridinyl)heptane, 1-Chloro-4-propoxythioxanthone, 1-Hydroxy cyclohexyl phenyl ketone, 2,2-Di ethoxy acetophenone, 2,3,4,4′-Tetrahydroxy Benzophenone, 2,3,4-Trihydroxybenzophenone, 2,4,6-Trimethyl benzoyl diphenyl phosphine oxide, 2,4,6-Trimethylbenzophenone, 2/4-Diethylthioxanthone, 2/4-Isopropylthioxanthone, 2-Benzyl-2-(dimethylamino)-1-[4-(4-morpholinyl)phenyl]-1-butanone, 2-Chlorothioxanthone, 2-Dimethyl-aminoethylbenzoate, 2-Ethylhexyl-4-dimethylaminobenzoate, 2-Hydroxy-2-methyl-phenyl-propan-1-one, 2-Hydroxy-4′-hydroxyethoxy-2-methylpropiophenone, 2-Isopropylthioxanthone, 2-Methyl benzophenone, 2-Methyl-1-[4-(methylthio)phenyl]-2-morpholinopropanone-1,4-(4-Methylphen-ylthiophenyl)-phenylmethanone, 4,4′-Difluoro benzophenone, 4,4′-Dimethoxy benzophenone, 4-Chloro benzophenone, 4-Methyl acetophenone, 4-Methyl benzophenone, 4-Phenylbenzophenone, Benzil dimethyl ketal, Benzophenone, Benzophenone hydrazone, Bis(p-tolyl) iodonium hexafluorophosphate, Dimethyl Sebacate, Diphenyl lodonium Hexafluorophosphate, Ethyl (2,4,6-trimethylbenzoyl)phenylphosphinate, Ethyl-4-(dimethylamino)benzoate, Methyl o-benzoyl benzoate, Methyl phenyl glyoxylate, N,N,N′,N′-Tetraethyl-4,4-diaminobenzophenone, Phenyltribromomethylsulphone, acylphosphine oxide (APO) and bisacylphosphine oxide (BAPO), 1-[4-(2-Hydroxyethoxy)-phenyl]-2-hydroxy-2-methyl-1-propane-1-one, 2,2-Dimethoxy-1,2-diphenylethan-1-one, hydroxy-cyclohexyl-phenyl-ketone, methylbenzoylformate, oxy-phenyl-acetic acid 2-[2 oxo-2 phenyl-acetoxy-ethoxy]ethyl ester, oxy-phenyl-acetic2-[2-hydroxy-ethoxy]-ethyl ester, alpha-dimethoxy-alpha-phenylacetophenone, 2-Benzyl-2-(dimethylamino)-1-[4-(4-morpholinyl)phenyl]-1-butanone, diphenyl (2,4,6-trimethylbenzoyl)-phosphine oxide, phosphine oxide, bis(eta 5-2,4-cyclopentadien-1-yl), bis[2,6-difluoro-3-(1H-pyrrol-1-yl)phenyl]titanium, Iodonium, (4-methylphenyl)[4-(2-methylpropyl)phenyl]-hexafluorophosphate(1-), bis(2,6-dimethoxybenz-oyl)-2,4,4-trimethyl pentylphosphineoxide, or derivatives thereof.

In embodiments, the photoinitiator is riboflavin. In embodiments, the riboflavin is riboflavin 5′ monophosphate sodium salt. In embodiments, the semi-gel composition comprises about 0.001 wt % to about 0.01 wt % riboflavin, e.g. about 0.0010 wt %, about 0.0015 wt %, about 0.0020 wt %, about 0.0025 wt %, about 0.0030 wt %, about 0.0035 wt %, about 0.0040 wt %, about 0.0045 wt %, about 0.0050 wt %, about 0.0055 wt %, about 0.0060 wt %, about 0.0065 wt %, about 0.0070 wt %, about 0.0075 wt %, about 0.0080 wt %, about 0.0085 wt %, about 0.0090 wt %, about 0.0095 wt %, or about 0.0100 wt %, including all ranges and values in between. In embodiments, the semi-gel composition comprises about 0.004 wt % riboflavin. In embodiments, the riboflavin is riboflavin 5′ monophosphate sodium salt.

In embodiments, the semi-gel composition comprises about 0 wt % to about 0.5 wt % arginine, e.g., about 0 wt %, about 0.10 wt %, about 0.15 wt %, about 0.20 wt %, about 0.25 wt %, about 0.30 wt %, about 0.35 wt %, about 0.40 wt %, about 0.45 wt %, or about 0.50 wt %, including all ranges and values in between. In embodiments, the semi-gel composition comprises about 0.3 wt % arginine. In embodiments, the arginine is L-arginine.

In embodiments, the semi-gel composition comprises about 97 wt % water, about 2 wt % methacrylated chitosan, about 1 wt % hyaluronic acid, about 0.3 wt % L-arginine, and about 0.004 wt % riboflavin 5′ monophosphate sodium salt.

In embodiments, the viscosity of the semi-gel composition is about 0.01 kPa*s to about 1 kPa*s, as measured by rheometry, e.g., about 0.01 kPa*s, about 0.02 kPa*s, about 0.03 kPa*s, about 0.04 kPa*s, about 0.05 kPa*s, about 0.06 kPa*s, about 0.07 kPa*s, about 0.08 kPa*s, about 0.09 kPa*s, about 0.1 kPa*s, about 0.2 kPa*s, about 0.3 kPa*s, about 0.4 kPa*s, about 0.5 kPa*s, about 0.6 kPa*s, about 0.7 kPa*s, about 0.8 kPa*s, about 0.9 kPa*s, or about 1.0 kPa*s, including all ranges and values in between. In embodiments, the viscosity of the semi-gel composition is about 0.01 kPa*s to about 1.0 kPa*s, as measured by rheometry. In embodiments, the viscosity of the semi-gel composition is about 0.01 kPa*s to about 0.3 kPa*s, as measured by rheometry. In embodiments, the viscosity of the semi-gel composition is about 0.5 kPa*s to about 1.0 kPa*s, as measured by rheometry.

In embodiments, the semi-gel composition is sterilized, wherein the sterilization is achieved with ionizing radiation. In embodiments, the ionizing radiation is electron beam radiation. In embodiments, the ionizing radiation is x-ray radiation. In embodiments, the ionizing radiation is gamma radiation.

In embodiments, sterilization partially cures the composition. In embodiments, sterilization is required to fully cure the composition. In embodiments, e-beam radiation partially cures the gel. In embodiments, x-ray radiation partially cures the gel. In embodiments, gamma irradiation partially cures the composition.

In embodiments, the dose of ionizing radiation is about 10 kGy to about 70 kGy, about 10 kGy, about 12 kGy, about 14 kGy, about 16 kGy, about 18 kGy, about 20 kGy, about 22 kGy, about 24 kGy, about 25 kGy, about 26 kGy, about 28 kGy, about 30 kGy, about 32 kGy, about 34 kGy, about 36 kGy, about 38 kGy, about 40 kGy, about 42 kGy, about 44 kGy, about 46 kGy, about 48 kGy, about 50 kGy, about 52 kGy, about 54 kGy, about 56 kGy, about 58 kGy, about 60 kGy, about 62 kGy, about 64 kGy, about 66 kGy, about 68 kGy, or about 70 kGy, including all ranges and values in between. In embodiments, the dose of ionizing radiation is about 25 kGy.

In embodiments, after sterilization, there is a log reduction of between about 2 and about 4 in colony forming units, e.g. about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, or about 5.0, including all ranges and values in between. In embodiments, after sterilization, there is a log reduction of between about 3.4 and about 3.6 in colony forming units. In embodiments, after sterilization, there is a log reduction of about 3.4 in colony forming units.

In embodiments, the semi-gel adheres to epithelial and mucosal tissue with strengths ranging from about 1 kPa to about 20 kPa, as measured using lap shear tests. In embodiments, the semi-gel adheres to epithelial and mucosal tissue with strengths ranging from about 0.1 kPa to about 20 kPa, as measured using lap shear tests, e.g., about 0.1 kPa, about 0.2 kPa, about 0.3 kPa, about 0.4 kPa, about 0.5 kPa, about 0.6 kPa, about 0.7 kPa, about 0.8 kPa, about 0.9 kPa, about 1.0 kPa, about 1.5 kPa, about 2.0 kPa, about 2.5 kPa, about 3.0 kPa, about 3.5 kPa, about 4.0 kPa, about 4.5 kPa, about 5.0 kPa, about 5.5 kPa, about 6.0 kPa, about 6.5 kPa, about 7.0 kPa, about 7.5 kPa, about 8.0 kPa, about 8.5 kPa, about 9.0 kPa, about 9.5 kPa, about 10.0 kPa, about 10.5 kPa, about 11.0 kPa, about 11.5 kPa, about 12.0 kPa, about 12.5 kPa, about 13.0 kPa, about 13.5 kPa, about 14.0 kPa, about 14.5 kPa, about 15.0 kPa, about 15.5 kPa, about 16.0 kPa, about 16.5 kPa, about 17.0 kPa, about 17.5 kPa, about 18.0 kPa, about 18.5 kPa, about 19.0 kPa, about 19.5 kPa, or about 20.0 kPa, including all ranges and values in between.

In embodiments, the semi-gel is a shear-thinning thixotropic material.

### Cured Composition

Compositions according to the present disclosure include cured compositions, which are composition that are cured resulting in a polymer matrix. In embodiments, cured composition has a sufficient stiffness and/or tackiness (for example as a result of functional groups providing cell adhesion domains) such that epithelial cells in the TM can migrate into and/or onto the scaffold and adhere and/or grow. In embodiments, the composition of the present disclosure is a cured composition produced by photoirradiation. In embodiments, the cured composition is prepared from photoirradiation of the semi-gel composition.

In embodiments, the viscosity or stiffness of the composition of the present disclosure is described by elastic modulus as measured by rheometry. In embodiments, the elastic modulus can be measured as described in the literature. For example, the elastic modulus can be measured using a parallel plate rheometer test that exerts oscillating shear force on the composition.

In embodiments, the elastic modulus of the cured composition is about 0.05 kPa to about 100 kPa as measured by rheometry, e.g., about 0.05 kPa, about 0.10 kPa, about 0.15 kPa, about 0.20 kPa, about 0.25 kPa, about 0.30 kPa, about 0.35 kPa, about 0.40 kPa, about 0.45 kPa, about 0.50 kPa, about 0.55 kPa, about 0.60 kPa, about 0.65 kPa, about 0.70 kPa, about 0.75 kPa, about 0.80 kPa, about 0.85 kPa, about 0.90 kPa, about 0.95 kPa, about 1.0 kPa, about 1.5 kPa, about 2.0 kPa, about 2.5 kPa, about 3.0 kPa, about 3.5 kPa, about 4.0 kPa, about 4.5 kPa, about 5.0 kPa, about 5.50 kPa, about 6.0 kPa, about 6.5 kPa, about 7.0 kPa, about 7.5 kPa, about 8.0 kPa, about 8.5 kPa, about 9.0 kPa, about 9.5 kPa, about 10 kPa, about 11 kPa, about 12 kPa, about 13 kPa, about 14 kPa, about 15 kPa, about 16 kPa, about 17 kPa, about 18 kPa, about 19 kPa, about 20 kPa, about 21 kPa, about 22 kPa, about 23 kPa, about 24 kPa, about 25 kPa, about 26 kPa, about 27 kPa, about 28 kPa, about 29 kPa, about 30 kPa, about 31 kPa, about 32 kPa, about 33 kPa, about 34 kPa, about 35 kPa, about 36 kPa, about 37 kPa, about 38 kPa, about 39 kPa, about 40 kPa, about 41 kPa, about 42 kPa, about 43 kPa, about 44 kPa, about 45 kPa, about 46 kPa, about 47 kPa, about 48 kPa, about 49 kPa, about 50 kPa, about 51 kPa, about 52 kPa, about 53 kPa, about 54 kPa, about 55 kPa, about 56 kPa, about 57 kPa, about 58 kPa, about 59 kPa, about 60 kPa, about 61 kPa, about 62 kPa, about 63 kPa, about 64 kPa, about 65 kPa, about 66 kPa, about 67 kPa, about 68 kPa, about 69 kPa, about 70 kPa, about 71 kPa, about 72 kPa, about 73 kPa, about 74 kPa, about 75 kPa, about 76 kPa, about 77 kPa, about 78 kPa, about 79 kPa, about 80 kPa, about 81 kPa, about 82 kPa, about 83 kPa, about 84 kPa, about 85 kPa, about 86 kPa, about 87 kPa, about 88 kPa, about 89 kPa, about 90 kPa, about 91 kPa, about 92 kPa, about 93 kPa, about 94 kPa, about 95 kPa, about 96 kPa, about 97 kPa, about 98 kPa, about 99 kPa, or about 100 kPa, including all ranges and values in between. In embodiments, the elastic modulus of the cured composition is about 0.4 kPa to about 3 kPa as measured by rheometry.

In embodiments, the cured composition comprises a diluent, methacrylated chitosan, hyaluronic acid, and a photoinitiator. In embodiments, the cured composition further comprises arginine. In embodiments, the arginine is L-arginine. In embodiments, the photoinitiator is riboflavin. In embodiments, the riboflavin is riboflavin 5′ monophosphate sodium salt. In embodiments, the diluent comprises water. In embodiments, the cured composition comprises about 80 wt % to about 99 wt % diluent, 1 wt % to about 3 wt % methacrylated chitosan, 0.5 wt % to about 2 wt % hyaluronic acid, 0.001 wt % to about 0.01 wt % riboflavin, and 0 wt % to about 0.5 wt % arginine.

In embodiments, the cured composition comprises about 80 wt % to about 99 wt % diluent, e.g about 80 wt %, about 81 wt %, about 82 wt %, about 83 wt %, about 84 wt %, about 85 wt %, about 86 wt %, about 87 wt %, about 88 wt %, about 89 wt %, about 90 wt %, about 91 wt %, about 92 wt %, about 93 wt %, about 94 wt %, about 95 wt %, about 96 wt %, about 97 wt %, about 98 wt %, or about 99 wt %, including all ranges and values in between. In embodiments, the semi-gel composition comprises about 90 wt % to about 97 wt % water. In embodiments, the semi-gel composition comprises about 97 wt % water. In embodiments, the diluent comprises water. In embodiments, the diluent comprises water and sodium chloride. In embodiments, the diluent comprises water and phosphate buffer. In embodiments, the diluent comprises water, sodium chloride and phosphate buffer. In embodiments the diluent is 0.9% sodium chloride in water (i.e., saline).

In embodiments, the cured composition comprises about 1 wt % to about 3 wt % methacrylated chitosan, e.g. about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2 wt %, about 2.1 wt %, about 2.2 wt %, about 2.3 wt %, about 2.4 wt %, about 2.5 wt %, about 2.6 wt %, about 2.7 wt %, about 2.8 wt %, about 2.9 wt %, or about 3 wt %, including all ranges and values in between. In embodiments, the semi-gel composition comprises about 2 wt % methacrylated chitosan. In embodiments, the methacrylated chitosan is about 25% to about 40% methacrylated.

In embodiments, the cured composition comprises about 0.5 wt % to about 2 wt % hyaluronic acid, e.g. about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, or about 2 wt %, including all ranges and values in between. In embodiments, the semi-gel composition comprises about 1 wt % hyaluronic acid.

In embodiments, the photoinitiator is an aromatic carbonyl compound (e.g., benzoin derivatives, benziketals, acetophenone derivatives, hydroxyalkylphenones) or an aromatic ketone (e.g., benzophenone and thioxanthone). In embodiments, the photoinitiator is benzophenone, dimethoxyphenyl acetophenone, 2,2-dimethoxy-2-phenylacetophenone and 2,2-diethoxyacetophenone, 1-[4-(2-hydroxyethoxy)-phenyl]-2-hydroxy-2-methyl-1-propane-1-one, ethyl eosin, eosin Y, fluorescein, 2,2-dimethoxy-2-phenylacetophenone, 2-methyl-2-phenylacetonphenone, 12959, camphorquinone, rose bengal, methylene blue, erythosin, phloxime, thionine, riboflavin, and methyl green. Still other photoinitiators comprise 1-(4-Fluorphenyl)-2-methyl-2-morpholino-1-propanone, 1,7-bis(9-acridinyl)heptane, 1-Chloro-4-propoxythioxanthone, 1-Hydroxy cyclohexyl phenyl ketone, 2,2-Di ethoxy acetophenone, 2,3,4,4′-Tetrahydroxy Benzophenone, 2,3,4-Trihydroxybenzophenone, 2,4,6-Trimethyl benzoyl diphenyl phosphine oxide, 2,4,6-Trimethylbenzophenone, 2/4-Diethylthioxanthone, 2/4-Isopropylthioxanthone, 2-Benzyl-2-(dimethylamino)-1-[4-(4-morpholinyl)phenyl]-1-butanone, 2-Chlorothioxanthone, 2-Dimethyl-aminoethylbenzoate, 2-Ethylhexyl-4-dimethylaminobenzoate, 2-Hydroxy-2-methyl-phenyl-propan-1-one, 2-Hydroxy-4′-hydroxyethoxy-2-methylpropiophenone, 2-Isopropylthioxanthone, 2-Methyl benzophenone, 2-Methyl-1-[4-(methylthio)phenyl]-2-morpholinopropanone-1,4-(4-Methylphen-ylthiophenyl)-phenylmethanone, 4,4′-Difluoro benzophenone, 4,4′-Dimethoxy benzophenone, 4-Chloro benzophenone, 4-Methyl acetophenone, 4-Methyl benzophenone, 4-Phenylbenzophenone, Benzil dimethyl ketal, Benzophenone, Benzophenone hydrazone, Bis(p-tolyl) iodonium hexafluorophosphate, Dimethyl Sebacate, Diphenyl lodonium Hexafluorophosphate, Ethyl (2,4,6-trimethylbenzoyl)phenylphosphinate, Ethyl-4-(dimethylamino)benzoate, Methyl o-benzoyl benzoate, Methyl phenyl glyoxylate, N,N,N′,N′-Tetraethyl-4,4-diaminobenzophenone, Phenyltribromomethylsulphone, acylphosphine oxide (APO) and bisacylphosphine oxide (BAPO), 1-[4-(2-Hydroxyethoxy)-phenyl]-2-hydroxy-2-methyl-1-propane-1-one, 2,2-Dimethoxy-1,2-diphenylethan-1-one, hydroxy-cyclohexyl-phenyl-ketone, methylbenzoylformate, oxy-phenyl-acetic acid 2-[2 oxo-2 phenyl-acetoxy-ethoxy]ethyl ester, oxy-phenyl-acetic2-[2-hydroxy-ethoxy]-ethyl ester, alpha-dimethoxy-alpha-phenylacetophenone, 2-Benzyl-2-(dimethylamino)-1-[4-(4-morpholinyl)phenyl]-1-butanone, diphenyl (2,4,6-trimethylbenzoyl)-phosphine oxide, phosphine oxide, bis(eta 5-2,4-cyclopentadien-1-yl), bis[2,6-difluoro-3-(1H-pyrrol-1-yl)phenyl]titanium, Iodonium, (4-methylphenyl)[4-(2-methylpropyl)phenyl]-hexafluorophosphate(1-), bis(2,6-dimethoxybenz-oyl)-2,4,4-trimethyl pentylphosphineoxide, or derivatives thereof.

In embodiments, the photoinitiator is riboflavin. In embodiments, the riboflavin is riboflavin 5′ monophosphate sodium salt. In embodiments, the cured composition comprises about 0.001 wt % to about 0.01 wt % riboflavin, e.g., about 0.0010 wt %, about 0.0015 wt %, about 0.0020 wt %, about 0.0025 wt %, about 0.0030 wt %, about 0.0035 wt %, about 0.0040 wt %, about 0.0045 wt %, about 0.0050 wt %, about 0.0055 wt %, about 0.0060 wt %, about 0.0065 wt %, about 0.0070 wt %, about 0.0075 wt %, about 0.0080 wt %, about 0.0085 wt %, about 0.0090 wt %, about 0.0095 wt %, or about 0.0100 wt %, including all ranges and values in between. In embodiments, the cured composition comprises about 0.004 wt % riboflavin. In embodiments, the riboflavin is riboflavin 5′ monophosphate sodium salt.

In embodiments, the cured composition comprises about 0 wt % to about 0.5 wt % arginine, e.g., about 0 wt %, about 0.10 wt %, about 0.15 wt %, about 0.20 wt %, about 0.25 wt %, about 0.30 wt %, about 0.35 wt %, about 0.40 wt %, about 0.45 wt %, or about 0.50 wt %, including all ranges and values in between. In embodiments, the semi-gel composition comprises about 0.3 wt % arginine. In embodiments, the arginine is L-arginine.

In embodiments, the cured composition comprises about 96.8 wt % water, about 1.936 wt % methacrylated chitosan, about 0.968 wt % hyaluronic acid, and about 0.29 wt % L-arginine, and 0.004 wt % riboflavin 5′ monophosphate sodium salt.

### Kits of the Present Disclosure

In embodiments, the present disclosure provides a kit comprising: a composition the present disclosure (e.g., lyophilized composition or semi-gel composition), a gel dispensing tray and a light guide. In embodiments, the kit is enclosed in a sealed tray.

In embodiments, the kit is sterilized after sealing the tray. In embodiments, the sterilization is achieved with ionizing radiation. In embodiments, the ionizing radiation is electron beam radiation. In embodiments, the ionizing radiation is x-ray radiation. In embodiments, the ionizing radiation is gamma radiation.

In embodiments, the dose of ionizing radiation is about 10 kGy to about 70 kGy, about 10 kGy, about 12 kGy, about 14 kGy, about 16 kGy, about 18 kGy, about 20 kGy, about 22 kGy, about 24 kGy, about 25 kGy, about 26 kGy, about 28 kGy, about 30 kGy, about 32 kGy, about 34 kGy, about 36 kGy, about 38 kGy, about 40 kGy, about 42 kGy, about 44 kGy, about 46 kGy, about 48 kGy, about 50 kGy, about 52 kGy, about 54 kGy, about 56 kGy, about 58 kGy, about 60 kGy, about 62 kGy, about 64 kGy, about 66 kGy, about 68 kGy, or about 70 kGy, including all ranges and values in between. In embodiments, the dose of ionizing radiation is about 25 kGy.

In embodiments, after sterilization, there is a log reduction of between about 2 and about 4 in colony forming units, e.g., about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, or about 5.0, including all ranges and values in between. In embodiments, after sterilization, there is a log reduction of between about 3.4 and about 3.6 in colony forming units. In embodiments, after sterilization, there is a log reduction of about 3.4 in colony forming units.

In embodiments, the sterilized composition (e.g., the sterilized lyophilized composition or sterilized semi-gel composition) is contained within a pouch. In embodiments, the lyophilized composition or semi-gel composition) is contained within a pouch. In embodiments, the pouch comprises a moisture barrier. In embodiments, the pouch comprises a transparent section. In embodiments, the pouch comprises a moisture barrier and a transparent section. In embodiments, the pouch comprises a luer lock connector. In embodiments, the pouch has an area of about 9 cm² to about 30 cm², e.g., about 9 cm², about 10 cm², about 11 cm², about 12 cm², about 13 cm², about 14 cm², about 15 cm², about 16 cm², about 17 cm² about 18 cm², about 19 cm², about 20 cm², about 21 cm², about 22 cm², about 23 cm², about 24 cm², about 25 cm², about 26 cm², about 27 cm², about 28 cm², about 29 cm², or about 30 cm², including all ranges and values in between.

In embodiments, the pouch comprises about 0.2 mL to about 10 mL of the composition, e.g., about 0.2 mL, about 0.3 mL, about 0.4 mL, about 0.5 mL, about 0.6 mL, about 0.7 mL, about 0.8 mL, about 0.9 mL, about 1.0 mL, about 1.1 mL, about 1.2 mL, about 1.3 mL, about 1.4 mL, about 1.5 mL, about 1.6 mL, about 1.7 mL, about 1.8 mL, about 1.9 mL, about 2 mL, about 3 mL, about 4 mL, about 5 mL, about 6 mL, about 7 mL, about 8 mL, about 9 mL, or about 10 mL, including all ranges and values in between.

In embodiments, the composition is contained within a mixing vessel (e.g., a syringe, a sachet, vial or a tube). In embodiments, the composition is contained within a mixing syringe.

In embodiments, the kit has a shelf life greater than about 1 year. In embodiments, the kit has a shelf life of about 1 year.

In embodiments, the present disclosure provides a kit comprising: a lyophilized composition, a gel dispensing tray, and a light guide. In embodiments, the lyophilized composition is contained within a pouch. In embodiments, the kit is enclosed in a sealed tray.

### Methods of the Present Disclosure

The present disclosure provides compositions and methods for use in treating perforations of the tympanic membrane. In embodiments, the perforations are chronic perforations of the tympanic membrane. In embodiments, the otologic materials comprise a semi-gel composition that is cured in situ resulting in a polymer matrix (e.g., a hydrogel scaffold). In embodiments, the methods comprise administering the semi-gel to a desired site in a patient's ear and curing the materials in situ. In embodiments, the methods comprise administering the semi-gel composition to an otologic packing material and curing the semi-gel composition after administration to the packing material.

In embodiments, the present disclosure provides a method of treating a perforation of the tympanic membrane comprising:
(i) applying a semi-gel composition of the present disclosure to the tympanic membrane,
(ii) inserting the light guide into the ear canal, and
(iii) curing the composition with photoirradiation.

In embodiments, the photoirradiation comprises irradiation of the composition with a wavelength of about 200 nm to about 800 nm, a curing time of about 1 seconds to about 300 seconds and a photoirradiation intensity of about 100 mW/cm² to about 100000 mW/cm².

In embodiments, about 50 μL to about 500 μL of the composition is applied to the tympanic membrane. In embodiments, about 10 μL to about 500 μL of the composition is applied to the tympanic membrane, e.g. about 10 μL, about 20 μL, about 30 μL, about 40 μL, about 50 μL, about 60 μL, about 70 μL, about 80 μL, about 90 μL, about 100 μL, about 120 μL, about 140 μL, about 160 μL, about 180 μL, about 200 μL, about 220 μL, about 240 μL, about 260 μL, about 280 μL, about 300 μL, about 320 μL, about 340 μL, about 360 μL, about 380 μL, about 400 μL, about 420 μL, about 440 μL, about 460 μL, about 480 μL, or about 500 μL, including all ranges and values in between. In embodiments, about 200 μL of the composition is applied to the tympanic membrane.

In embodiments, the photoirradiation comprises irradiation of semi-gel composition with a wavelength of about 200 nm to about 800 nm, a curing time of about 1 seconds to about 300 seconds, and a photoirradiation intensity of about 100 mW/cm² to about 1000 mW/cm². In embodiments, the photoirradiation comprises irradiation of semi-gel composition with a wavelength of about 200 nm to about 800 nm, a curing time of about 1 seconds to about 300 seconds, and a photoirradiation intensity of about 100 mW/cm² to about 10000 mW/cm². In embodiments, the photoirradiation comprises irradiation of semi-gel composition with a wavelength of about 200 nm to about 800 nm, a curing time of about 1 seconds to about 300 seconds, and a photoirradiation intensity of about 100 mW/cm² to about 100000 mW/cm². In embodiments, the photoirradiation comprises irradiation of a composition (e.g., semi-gel composition) with a wavelength of about 200 nm to about 800 nm, a curing time of about 1 seconds to about 300 seconds and a photoirradiation power of about 2 mW to about 1 W. In embodiments, the photoirradiation comprises irradiation of a composition (e.g., semi-gel composition) with a wavelength of about 200 nm to about 800 nm, a curing time of about 1 seconds to about 300 seconds and a photoirradiation power of about 0.2 mW to about 1 W.

In embodiments, the wavelength is about 200 nm to about 800 nm, e.g. about 200 nm, about 205 nm, about 210 nm, about 215 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 280 nm, about 285 nm, about 290 nm, about 295 nm, about 300 nm, about 305 nm, about 310 nm, about 315 nm, about 320 nm, about 325 nm, about 330 nm, about 335 nm, about 340 nm, about 345 nm, about 350 nm, about 355 nm, about 360 nm, about 365 nm, about 370 nm, about 375 nm, about 380 nm, about 385 nm, about 390 nm, about 395 nm, about 400 nm, about 405 nm, about 410 nm, about 415 nm, about 420 nm, about 425 nm, about 430 nm, about 435 nm, about 440 nm, about 445 nm, about 450 nm, about 455 nm, about 460 nm, about 465 nm, about 470 nm, about 475 nm, about 480 nm, about 485 nm, about 490 nm, about 495 nm, about 500 nm, about 505 nm, about 510 nm, about 515 nm, about 520 nm, about 525 nm, about 530 nm, about 535 nm, about 540 nm, about 545 nm, about 550 nm, about 555 nm, about 560 nm, about 565 nm, about 570 nm, about 575 nm, about 580 nm, about 585 nm, about 590 nm, about 595 nm, about 600 nm, about 605 nm, about 610 nm, about 615 nm, about 620 nm, about 625 nm, about 630 nm, about 635 nm, about 640 nm, about 645 nm, about 650 nm, about 655 nm, about 660 nm, about 665 nm, about 670 nm, about 675 nm, about 680 nm, about 685 nm, about 690 nm, about 695 nm, about 700 nm, about 705 nm, about 710 nm, about 715 nm, about 720 nm, about 725 nm, about 730 nm, about 735 nm, about 740 nm, about 745 nm, about 750 nm, about 755 nm, about 760 nm, about 765 nm, about 770 nm, about 775 nm, about 780 nm, about 785 nm, about 790 nm, about 795 nm, or about 800 nm, including all ranges and values in between. In embodiments, the irradiation wavelength is about 420 nm to about 530 nm. In embodiments, the irradiation wavelength is about 450 nm.

In embodiments, the photoirradiation power is about 2 mW to about 1 W. In embodiments, the photoirradiation power is about 0.2 mW to about 1 W, e.g., about 0.2 mW, about 0.4 mW, about 0.6 mW, about 0.8 mW, about 1.0 mW, about 1.2 mW, about 1.4 mW, about 1.6 mW, about 1.8 mW, about 2 mW, about 4 mW, about 5 mW, about 6 mW, about 7 mW, about 8 mW, about 9 mW, about 10 mW, about 11 mW, about 12 mW, about 14 mW, about 16 mW, about 18 mW, about 20 mW, about 22 mW, about 24 mW, about 26 mW, about 28 mW, about 30 mW, about 32 mW, about 34 mW, about 36 mW, about 38 mW, about 40 mW, about 42 mW, about 44 mW, about 46 mW, about 48 mW, about 50 mW, about 60 mW, about 70 mW, about 80 mW, about 90 mW, about 100 mW, about 110 mW, about 120 mW, about 130 mW, about 140 mW, about 150 mW, about 160 mW, about 170 mW, about 180 mW, about 190 mW, about 200 mW, about 300 mW, about 400 mW, about 500 mW, about 600 mW, about 700 mW, about 800 mW, about 900 mW, or about 1000 mW, including all ranges and values in between. In embodiments the photoirradiation power is about 15 mW to about 50 mW. In embodiments the irradiation power is about 25 mW.

In embodiments, the photoirradiation intensity is about 100 mW/cm² to about 1000 mW/cm². In embodiments, the photoirradiation intensity is about 100 mW/cm² to about 10000 mW/cm². In embodiments, the photoirradiation intensity is about 100 mW/cm² to about 100000 mW/cm², e.g. about 100 mW/cm², about 110 mW/cm², about 120 mW/cm², about 130 mW/cm², about 140 mW/cm², about 150 mW/cm², about 160 mW/cm², about 170 mW/cm², about 180 mW/cm², about 190 mW/cm², about 200 mW/cm², about 220 mW/cm², about 240 mW/cm², about 260 mW/cm², about 280 mW/cm², about 300 mW/cm², about 320 mW/cm², about 340 mW/cm², about 360 mW/cm², about 380 mW/cm², about 400 mW/cm², about 420 mW/cm², about 440 mW/cm², about 460 mW/cm², about 480 mW/cm², about 500 mW/cm², about 550 mW/cm², about 600 mW/cm², about 650 mW/cm², about 700 mW/cm², about 750 mW/cm², about 800 mW/cm², about 850 mW/cm², about 900 mW/cm², about 950 mW/cm², about 1000 mW/cm², about 1100 mW/cm², about 1200 mW/cm², about 1300 mW/cm², about 1400 mW/cm², about 1500 mW/cm², about 1600 mW/cm², about 1700 mW/cm², about 1800 mW/cm², about 1900 mW/cm², about 2000 mW/cm², about 2200 mW/cm², about 2400 mW/cm², about 2600 mW/cm², about 2800 mW/cm², about 3000 mW/cm², about 3200 mW/cm², about 3400 mW/cm², about 3600 mW/cm², about 3800 mW/cm², about 4000 mW/cm², about 5000 mW/cm², about 6000 mW/cm², about 7000 mW/cm², about 8000 mW/cm², about 9000 mW/cm², about 10000 mW/cm², about 11000 mW/cm², about 12000 mW/cm², about 13000 mW/cm², about 14000 mW/cm², about 15000 mW/cm², about 16000 mW/cm², about 17000 mW/cm², about 18000 mW/cm², about 19000 mW/cm², about 20000 mW/cm², about 30000 mW/cm², about 40000 mW/cm², about 50000 mW/cm², about 60000 mW/cm², about 70000 mW/cm², about 80000 mW/cm², about 90000 mW/cm², or about 100000 mW/cm², including all ranges and values in between. In embodiments, the irradiation is at a power of about 178 mW/cm².

In embodiments, the curing time is about 1 seconds to about 300 seconds (sec), e.g. about 1 sec, about 2 sec, about 3 sec, about 4 sec, about 5 sec, about 6 sec, about 7 sec, about 8 sec, about 9 sec, about 10 sec, about 12 sec, about 14 sec, about 16 sec, about 18 sec, about 20 sec, about 22 sec, about 24 sec, about 26 sec, about 28 sec, about 30 sec, about 32 sec, about 34 sec, about 36 sec, about 38 sec, about 40 sec, about 42 sec, about 44 sec, about 46 sec, about 48 sec, about 50 sec, about 52 sec, about 54 sec, about 56 sec, about 58 sec, about 60 sec, about 65 sec, about 70 sec, about 75 sec, about 80 sec, about 85 sec, about 90 sec, about 95 sec, about 100 sec, about 110 sec, about 120 sec, about 130 sec, about 140 sec, about 150 sec, about 160 sec, about 170 sec, about 180 sec, about 190 sec, about 200 sec, about 210 sec, about 220 sec, about 230 sec, about 240 sec, about 250 sec, about 260 sec, about 270 sec, about 280 sec, about 290 sec, or about 300 sec, including all ranges and values in between. In embodiments, the curing time is about 5 seconds to about 120 seconds. In embodiment, the curing time is about 12 seconds to about 300 seconds. In embodiments, the curing time is 90 seconds.

In embodiments, the photoirradiation (i.e., curing time) is continuous. In embodiments, the photoirradiation (i.e., curing time) is administered as one or more bursts, e.g., 1 burst, 2 bursts, 3 bursts, 4 bursts or 5 bursts. For example, in embodiments, the curing time is about 90 seconds and the photoirradiation is administered as three bursts of about 30 seconds each. In embodiments, the photoirradiation is administered in one burst. In embodiments, the photoirradiation is administered in two bursts. In embodiments, the photoirradiation is administered as three bursts. In embodiments, the time between bursts is about 5 seconds to about 30 seconds, e.g., about 1 sec, about 2 sec, about 3 sec, about 4 sec, about 5 sec, about 6 sec, about 7 sec, about 8 sec, about 9 sec, about 10 sec, about 12 sec, about 14 sec, about 16 sec, about 18 sec, about 20 sec, about 22 sec, about 24 sec, about 26 sec, about 28 sec, or about 30 sec, including all ranges and values in between. In embodiments, the time between bursts is about 10 sec.

In embodiments, the present disclosure provides a method of treating a perforation of the tympanic membrane in a patient in need thereof comprising: (a) adding and mixing a solvent (such as saline) with any one of the lyophilized compositions of the present disclosure to form a semi-gel composition, (b) administering the semi-gel composition to the perforation site in the tympanic membrane of the patient, and (c) curing the applied semi-gel composition with photoirradiation. In embodiments, the lyophilized composition is mixed with about 0.1 mL to about 2 mL of a solvent (such as saline), e.g., about 0.1 mL, about 0.2 mL, about 0.3 mL, about 0.4 mL, about 0.5 mL, about 0.6 mL, about 0.7 mL, about 0.8 mL, about 0.9 mL, about 1.0 mL, about 1.1 mL, about 1.2 mL, about 1.3 mL, about 1.4 mL, about 1.5 mL, about 1.6 mL, about 1.7 mL, about 1.8 mL, about 1.9 mL, or about 2.0 mL, including all values or ranges therebetween. In embodiments, the lyophilized composition is mixed with about 1 mL of saline. In embodiments, the semi-gel composition is directly applied to the perforation site in the tympanic membrane of the patient where it is cured by any one of the photoirradiation methods as described in the present disclosure. In embodiments, the cured semi-gel composition provides a scaffold for endogenous cell migration and is replaced by newly formed tympanic membrane tissue.

In embodiments, the patient in need thereof is a child. In embodiments, the patient in need thereof is an adolescent 18 years or age or younger. In embodiments, the patient in need thereof is an adult. In embodiments, the patient is 5 years old or older or about at least 5 years old. In embodiments, the patient is 5-11 years old, e.g., about 5 years old, about 6 years old, about 7 years old, about 8 years old, about 9 years old, about 10 years old, or about 11 years old, including all values or ranges therebetween.

In embodiments, the method of the present disclosure provides a perforation treatment for the patient in need thereof, wherein the patient has at least about 1% (e.g., at least about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, including any values or ranges therebetween) of tympanic membrane affected by perforation. In embodiments, the method of the present disclosure provides a perforation treatment for the patient in need thereof, wherein the patient has at least about 5% of tympanic membrane affected by perforation. In embodiments, the method of the present disclosure provides a perforation treatment for the patient in need thereof, wherein the patient has at least about 10% of tympanic membrane affected by perforation. In embodiments, the patient has at least about 15% of tympanic membrane affected by perforation. In embodiments, the patient has at least about 20% of tympanic membrane affected by perforation. In embodiments, the patient has at least about 25% of tympanic membrane affected by perforation. In embodiments, the patient has at least about 30% of tympanic membrane affected by perforation. In embodiments, the patient has at least about 50% of tympanic membrane affected by perforation. In embodiments, the patient has at least about 80% of tympanic membrane affected by perforation.

In embodiments, the methods of the present disclosure provide at least about 40% tympanic membrane perforation closure rate. In embodiments, the methods of the present disclosure provide about ≥50% tympanic membrane perforation closure rate. In embodiments, the methods of the present disclosure provide about ≥70% tympanic membrane perforation closure rate. In embodiments, the methods of the present disclosure provide about ≥75% tympanic membrane perforation closure rate. In embodiments, the method of the present disclosure provides about >75% tympanic membrane perforation closure rate. In embodiments, the perforation closure rate is determined by visual assessment. In embodiments, a microscope or endoscope is used for the visual assessment.

In embodiments, the methods of the present disclosure improve about ≥40% in pure-tone average score and/or air-bone gap compared to baseline. In embodiments, the methods of the present disclosure provide about ≥50% improvement in pure-tone average score compared to baseline. In embodiments, the methods of the present disclosure provide about ≥70% improvement in pure-tone average score compared to baseline. In embodiments, the methods of the present disclosure provide about ≥75% improvement in pure-tone average score compared to baseline.

In embodiments, the method provides >75% tympanic membrane perforation closure rate or pure-tone average score compared to baseline by a single application. In embodiments, the method increases tympanic membrane perforation closure rate in about 1 week, about 2 weeks, about 3 weeks, about 4 weeks, about 5 weeks, about 6 weeks, about 7 weeks, about 8 weeks, about 9 weeks, about 10 weeks, about 11 weeks, about 3 months, about 4 months, about 5 months, or about 6 months following administration. In embodiments, the treatment increases tympanic membrane perforation closure rate in about 3 months following administration.

### EXAMPLES

### Example 1: Sterilization of a Lyophilized Composition

### Procedure

A kit was sterilized by irradiation at a dosage of 25 kGy. Irradiated samples were incubated in culture media for 24 h and 1 week at 37° C. Samples were then placed in the extraction media in a biosafety cabinet, with the use of disinfected instruments. After extraction, an aliquot of the extraction medium was plated on selective agar media and incubated overnight at 37° C. for identification of various microorganisms (both bacteria and fungi) that are possibly present.

### Results

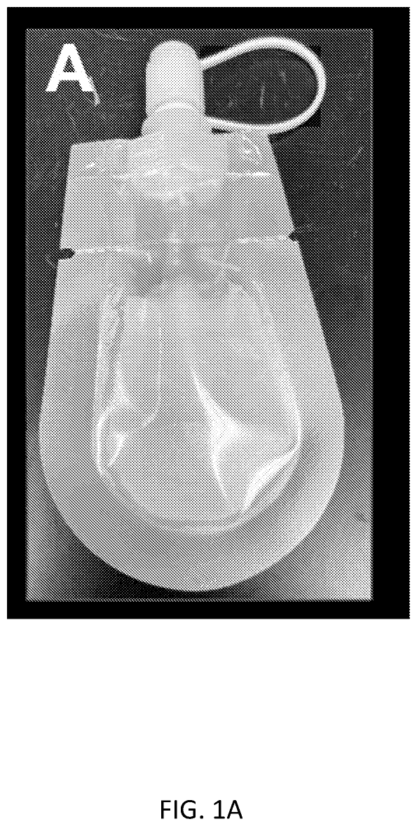

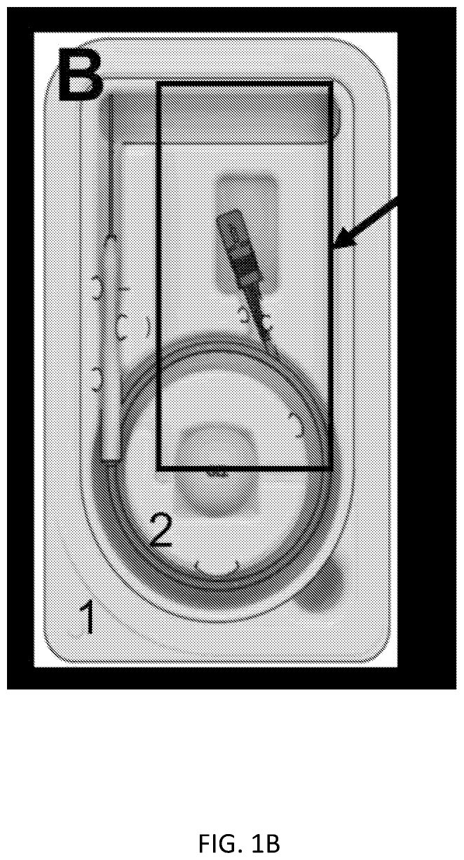

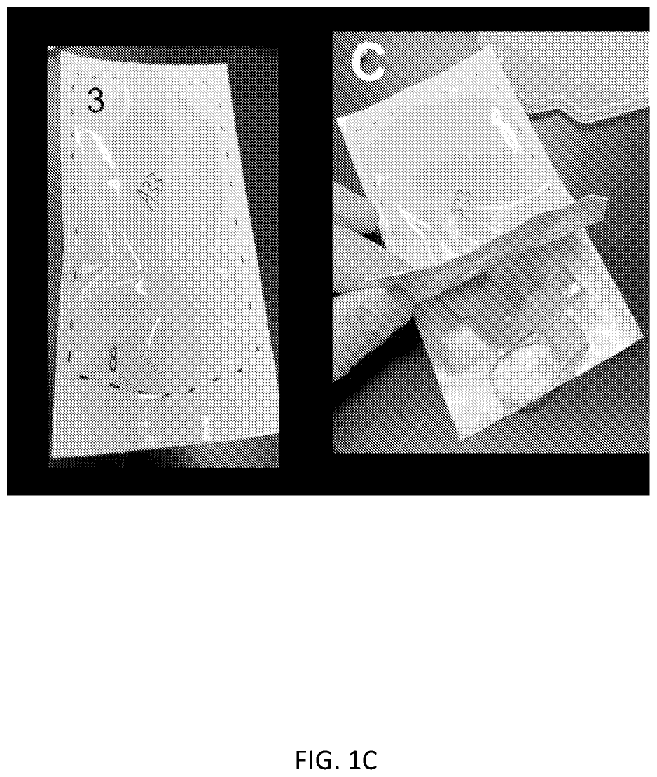

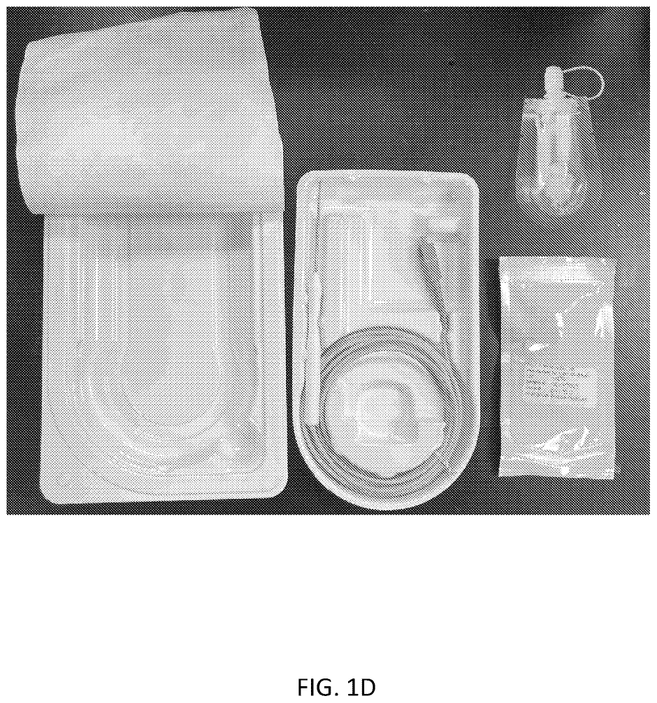

After 24 h and 1 week of incubation, no microbes were apparent on any of the selective media used for the test (FIGS. 1 and 2). In comparison, non-irradiated samples showed hundreds of colonies indicating the presence of bacteria. This suggested that the level of irradiation was sufficient to kill any microbes that may have been present pre-sterilization.

### Conclusion

Irradiation appears to be sufficient in sterilization. Compared to the non-irradiated control, the log reduction between unsterilized and sterilized samples was between 3.4 and 3.6.

### Example 2: Curing and Characterization of the Semi-gel

### Procedure

In an office setting, the lyophilized composition was rehydrated with 1 mL saline and mixed for 20-30 seconds to form a semi-gel composition. After preparing the treatment site, the semi-gel composition was applied to cover the tympanic membrane perforation with a 1 mm overlap of the margins. The semi-gel was irradiated for 1.5 min using a light guide fitted to a curing light.

### Results

Flow curve tests were conducted on the semi-gel at shear rates ranging from 0.01 s⁻¹ to 10 s⁻¹ on samples reconstituted in n=4 pouches. Viscosity values ranging from 0.01 kPa*s to 0.3 kPa*s at a shear rate of 1 s⁻¹ were obtained.

### Example 3: In Vivo Animal Model Establishment and Testing

### Procedure

Tympanic membranes of adult male chinchillas were perforated with a laser to create perforations of similar size. Perforations that remained open for 8 weeks were deemed chronic. A semi-gel composition was applied to the tympanic membrane perforations and cured with the curing light and light guide to form a cured composition.

### Results

Perforations healed within 3 weeks. Fourteen weeks after treatment with the cured composition, the tympanic membranes and cochlea were evaluated by histopathological analysis. Tympanic membrane perforations treated with the cured composition healed, and the regenerated tissue had the same architecture as native tympanic membrane tissue. Hair cells in the cochlea were intact, indicating that the cured composition was not ototoxic.

### Example 4: Evaluation of Ototoxic Potential of Cured Composition

### Procedure

Tympanic membranes of adult guinea pigs were perforated with a surgical knife then immediately treated with a semi-gel composition. The semi-gel composition was then cured using a light guide and curing light.

### Results

Perforations were monitored for 4 weeks, during which the perforations healed. Ototoxicity was evaluated by auditory brainstem responses (ABR) and distortion product otoacoustic emissions (DPOAE). Results from ABR and DPOAE tests indicated that hearing ability and hair cell integrity were not lost, indicating that the cured composition does not have any ototoxic potential.

### Example 5: Otologic Gel Kit

An exemplary otologic gel kit of the present disclosure is shown in FIGS. 1A-C. The components included in the kit are shown in Table 1.

TABLE 1
Otologic Gel Kit components
Part, Assembly, Technology, Software, Accessory

Otologic Gel Patch Gel Application Kit (Sterile)
1x Gel sachet containing the gel pouch
1x dispensing tray
1x light guide
Gel Curing Light Pack
1x Curing light
1x Foot pedal
1x Power cord
2x Safety glasses with care
Sterile saline
Clean application instrument

The otologic gel kit (i.e., otologic gel patch kit) provides a composition of the present disclosure comprising hyaluronic acid (HA), methacrylated chitosan (MeCS), and riboflavin monophosphate (RM). The composition is supplied in a pouch. The contents of the gel pouch are mixed with saline (1 mL of saline) prior to the time of application to a patient. After adding saline, the pouch is recapped then kneaded to mix the dry reagents with saline. After mixing, the end of the pouch is then torn off and the contents emptied into a supplied tray. The uncured composition is a semi-gel that is applied directly to the perforation through the external ear canal with a curette or other preferred instrument. Upon exposure to blue light from a curing light, the semi-gel of the composition forms a stiff, three-dimensional gel patch that fills the perforation site. Animal studies show the gel material remains in situ for less than 4 weeks.

### Example 6: Clinical Studies for Safety and Effectiveness of the Otologic Gel

The following example provides a clinical study to evaluate the safety and effectiveness of the compositions of the present disclosure in repairing chronic tympanic membrane perforation. The study will: i) assess the effectiveness of the composition as a gel patch in promoting the natural healing process to close chronic, ≥25% tympanic membrane perforation with a single application; and ii) assess safety of the composition and application procedure.

The study will also i) assess gel application procedural success (e.g., under variations in anatomical condition, perforation location, TMP size, and/or age of the patient); ii) measure time to apply the gel for a single application; iii) measure time from the gel patch application to tympanic membrane healing; iv) access changes in hearing in all patient population; v) assess the safety of the gel patch in the target population for the intended use; and vi) assess pain and discomfort using visual analogue scale (VAS).

The primary therapeutic endpoint will be to measure complete tympanic membrane perforation closure rates that are greater than 75% reported by clinical visual assessment at Visit 5 (3 months) following application of the gel composition. The safety endpoint of the incidence rate of device and procedure-related serious adverse events on and after administration will also be measured. Additional secondary endpoints include: complete tympanic membrane perforation closure assessed visually at Visit 5 after successful applications of gel composition with sedation; procedural success/failure rates; median time measured in minutes to successfully complete one application procedure; baseline and Visit 5 audiogram data comparisons of pure-tone average score and air-bone gap (ABG); baseline and Visit 5 word recognition sore data comparisons; baseline and Visit 5 tympanometry measurement data comparisons of tympanic membrane mobility; and median time measured in weeks to closure of tympanic membrane perforation after treatment.

Inclusion Criteria: Patients will be eligible for participation in this study if the patient met all of the following criteria at the Baseline visit:
1. Willing and able to provide informed consent, legally authorized representative (LAR) consent, and assent when age appropriate.
2. Females and males at least 5 years old.
3. Affected Ear Criteria:
   a. Perforation involves >25% of the tympanic membrane;
   b. Perforation has not spontaneously closed after 4 weeks of watchful waiting;
   c. Perforation is not actively healing;
   d. Perforation can be visualized by an endoscope or microscope; and
   e. Ear wax does not occlude the perforation.

Exclusion Criteria: Patients will be ineligible for participation in this study if the patient met any of the following criteria:
1. Perforation is marginal (a perforation that has an area with no tympanic membrane between the perforation and the bony canal).
2. Active otitis media, with or without effusion.
3. Otorrhea from the middle ear for more than 3 months.
4. History of cleft palate.
5. Receiving radiation therapy or taking corticosteroids, immunosuppressive agents, or chemotherapy.
6. Currently taking systemic antibiotics, antibiotic ear drops, and/or steroid ear drops.
7. Current bacterial or viral infection.
8. Fever (Temperature >100° F.) at time of index procedure.
9. Diagnosed with cholesteatoma mass in the tympanic cavity.
10. Known history of malignant ear canal tumors within 3 years of screening for eligibility.
11. Abrasions/lacerations to the external auditory canal.
12. Significant medical condition that could prevent full participation in the procedures required for the study.
13. Investigator feels the patient will be unable to cooperate with the application procedure.
14. Parent/LAR feels the patient will be unable to cooperate with the application procedure.
15. Allergy to shellfish.
16. Known to be or could be pregnant.
17. Adults lacking capacity to consent.

Study Design: Patients will undergo screening at Visit 1. Eligible patients will undergo treatment with the otologic gel applied at Visit 2 (Day 0) and will provide a post-procedure pain score. Perforations will be monitored at one week (Visit 3) and three weeks (Visit 4) post-procedure to determine perforation closure rate. At 12 weeks (Visit 5) following procedure, perforation closure and hearing ability will be evaluated. Audiometry and tympanometry tests will be performed at Visit 5 (i.e., end of study visit). Adverse events will be observed at each visit.

Otologic gel application procedure: The patient will be placed in a supine position on an exam room chair with their ear under a microscope or endoscope. The ENT clinician will administer uncured semi-gel composition material using a clean microsurgical tool. The semi-gel composition will be applied to the perforation so that there is approximately a 1-mm overlap of the uncured semi-gel composition and the TM tissue around the perforation. Since this procedure takes place under a microscope or endoscope, the ENT is able to be precise in covering the perforation with gel. The procedure is a clean procedure and does not require sterile application. The margins of the perforation will not be manipulated or abraded prior to application of the gel composition. Once the uncured gel composition material is applied and covers the perforation site, the ENT will cure the material with the provided reusable curing light and disposable light guide. The provided safety glasses will be worn by the patient, clinicians, and others present in the room. The curing light emits a beeping sound intermittently during the 90-second curing time period. The entire procedure to treat the ear should take about 10 minutes.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A pre-lyophilized composition comprising:
(i) a base comprising about 40 wt % to about 80 wt % methacrylated chitosan, about 20 wt % to about 40 wt % hyaluronic acid and about 0.01 wt % to about 0.5 wt % photoinitiator, 0 wt % to about 20 wt % arginine; and
(ii) a diluent.

2. The pre-lyophilized composition of claim 1, wherein the base comprises about 60 wt % methacrylated chitosan.

3. The pre-lyophilized composition of any one of claims 1 to 2, wherein the base comprises about 30 wt % hyaluronic acid.

4. The pre-lyophilized composition of any one of claims 1 to 3, wherein the base comprises about 0.12 wt % of a photoinitiator, wherein the photoinitator is riboflavin.

5. The pre-lyophilized composition of any one of claims 1 to 4, wherein the base comprises about 9 wt % arginine.

6. The pre-lyophilized composition of any one of claims 1 to 5, wherein the methacrylated chitosan is about 25% to about 40% methacrylated.

7. The pre-lyophilized composition of any one of claims 1 to 6, wherein the diluent comprises water, and wherein the diluent is about 0 wt % to about 95 wt % of the composition.

8. A lyophilized composition, comprising:
about 0 wt % to about 10 wt % water,
about 40 wt % to about 80 wt % methacrylated chitosan,
about 20 wt % to about 40 wt % hyaluronic acid,
about 0.01 wt % to about 0.5 wt % of a photoinitiator, and
about 0 wt % to about 20 wt % arginine.

9. The lyophilized composition of claim 8, wherein the composition comprises about 1 wt % water.

10. The lyophilized composition of claim 8 or 9, wherein the composition comprises about 60 wt % methacrylated chitosan.

11. The lyophilized composition of claim 8 or 10, wherein the methacrylated chitosan is about 25% to about 40% methacrylated.

12. The lyophilized composition of any one of claims 8 to 11, wherein the composition comprises about 30 wt % hyaluronic acid.

13. The lyophilized composition of any one of claims 8 to 12, wherein the composition comprises about 0.12 wt % photoinitator, wherein the photoinitator is riboflavin.

14. The lyophilized composition of any one of claims 8 to 13, wherein the composition comprises about 9 wt % arginine.

15. The lyophilized composition of any one of claims 8 to 14, wherein the composition can be reconstituted in less than 1 minute.

16. A semi-gel composition comprising:
about 80 wt % to about 99 wt % of a diluent,
about 1 wt % to about 3 wt % methacrylated chitosan,
about 0.5 wt % to about 2 wt % hyaluronic acid,
about 0.0010 wt % to about 0.01 wt % of a photoinitiator,
and about 0 wt % to about 0.5 wt % arginine.

17. The semi-gel composition of claim 16, wherein the composition comprises about 2 wt % methacrylated chitosan.

18. The semi-gel composition of claim 16 or 17, wherein the methacrylated chitosan is about 25% to about 40% methacrylated.

19. The semi-gel composition of any one of claims 16 to 18, wherein the composition comprises about 1 wt % hyaluronic acid.

20. The semi-gel composition of any one of claims 16 to 19, wherein the composition comprises about 0.004 wt % photoinitator, wherein the photoinitator is riboflavin.

21. The semi-gel composition of any one of claims 16 to 20, wherein the composition comprises about 0.3 wt % arginine.

22. The semi-gel composition of any one of claims 16 to 21, wherein the composition comprises about 97 wt % of a diluent.

23. The semi-gel composition of claim 16 or 22, wherein the diluent is saline.

24. The semi-gel composition of any one of claims 16 to 23, wherein the composition has a viscosity of about 0.01 kPa*s to about 0.3 kPa*s, as measured by rheometry.

25. The semi-gel composition of any one of claims 16 to 24, wherein the composition is prepared by mixing a lyophilized composition with a diluent until homogenous, and wherein the semi-gel composition is mixed with the lyophilized composition for a mixing time of less than about 60 seconds.

26. A cured composition comprising:
about 80 wt % to about 99 wt % of a diluent;
about 1 wt % to about 3 wt % methacrylated chitosan,
about 0.5 wt % to about 2 wt % hyaluronic acid,
about 0.0010 wt % to about 0.01 wt % of a photoinitiator; and
about 0 wt % to about 0.5 wt % arginine.

27. The cured composition of claim 26, wherein the composition comprises about 2 wt % methacrylated chitosan.

28. The cured composition of claim 26 or 27, wherein the methacrylated chitosan is about 25% to about 40% methacrylated.

29. The cured composition of any one of claims 26 to 28, wherein the composition comprises about 1 wt % hyaluronic acid.

30. The cured composition of any one of claims 26 to 29, wherein the composition comprises about 0.004 wt % photoinitator, wherein the photoinitator is riboflavin.

31. The cured composition of any one of claims 26 to 30, wherein the composition comprises about 0.3 wt % arginine.

32. The cured composition of any one of claims 26 to 31, wherein the composition comprises about 97 wt % of a diluent.

33. The cured composition of claim 26 or 32, wherein the diluent is saline.

34. The cured composition of any one of claims 26 to 33, wherein the composition has an elastic modulus of about 0.4 kPa to about 5 kPa, as measured by rheometry.

35. The cured composition of any one of claims 26 to 34, wherein the composition is prepared by photoirradiation of a semi-gel composition with a wavelength of about 200 nm to about 800 nm for a curing time of about 1 second to about 300 seconds at a photoirradiation intensity of about 100 mW/cm² to about 100000 mW/cm².

36. The cured composition of claim 35, wherein the photoirradiation comprises a curing time of about 90 seconds.

37. The composition of any one of claims 8 to 25, wherein the composition is sterilized with ionizing radiation.

38. The sterilized composition of claim 37, wherein the ionizing radiation is electron beam radiation.

39. The sterilized composition of claim 37 or 38, wherein after sterilization, there is a log reduction of between 3.4 and 3.6 in colony forming units.

40. A kit comprising: the composition of any one of claims 8 to 25, a gel dispensing tray, and a light guide.

41. The kit of claim 40, wherein the sterilized composition is contained within a pouch.

42. The kit of claim 40 or 41, wherein the kit is enclosed in a sealed tray.

43. The kit of any one of claims 40 to 42, wherein the pouch comprises a moisture barrier and a transparent section.

44. A method of treating a perforation of the tympanic membrane comprising:
(a). applying the semi-gel composition of any one of claims 16 to 25 to the tympanic membrane,
(b). inserting a light guide into the ear canal, and
(b). curing the composition with photoirradiation;
wherein the photoirradiation comprises irradiation of the composition with a wavelength of about 200 nm to about 800 nm, a curing time of about 1 seconds to about 300 seconds and a photoirradiation intensity of about 100 mW/cm² to about 100000 mW/cm².

45. The method of claim 44, wherein the curing time lasts about 90 seconds.

46. A method of treating a perforation of the tympanic membrane in a patient in need thereof comprising:
(a) adding and mixing saline with the lyophilized composition of any one of claim 8 to 15 to form a semi-gel composition;
(b) administering the semi-gel composition to the perforation site in the tympanic membrane of the patient in need thereof; and
(c) curing the applied semi-gel composition with photoirradiation.

47. The method of any one of claims 44-46, wherein the patient is 5 years old or older.

48. The method of claim 47, wherein the patient is 5-11 years old.

49. The method of any one of claims 44-48, the patient has at least about 25% of tympanic membrane affected by perforation.

50. The method of any one of claims 44-49, the patient has perforation in tympanic membrane for at least 4 weeks.

51. The method of any one of claims 44-50, wherein the lyophilized composition is mixed with about 0.1 mL to about 2 mL of saline.

52. The method of claim 51, wherein the lyophilized composition is mixed with about 1 mL of saline.

53. The method of any one of claims 44-52, wherein the curing time lasts about 90 seconds.

54. The method of any one of claims 44-53, wherein the cured semi-gel is biodegradable.

55. The method of any one of claims 44-54, wherein the cured semi-gel remains in the applied tympanic membrane for less than about 4 weeks.

56. The method of any one of claims 44-55, wherein the treatment provides about ≥75% tympanic membrane perforation closure rate.

57. The method of any one of claims 44-56, wherein the treatment provides ≥75% tympanic membrane perforation closure rate by a single application.

58. The method of any one of claims 44-57, wherein the treatment increases tympanic membrane perforation closure rate in about 3 months following administration.
